(19) United States
(12) Patent Application Publication
(10) Pub. No.: US 2008/0227847 A1
(43) Pub. Date: Sep. 18, 2008

(54) Novel Salts of Fumaric Acid Monoalkylesters and Their Pharmaceutical Use

(75) Inventors: Henrik Nilsson, Copenhagen (DK); Jens E.T. Andersen, Vedbaek (DK); Bernd W. Mueller, Flintbek (DE)

Correspondence Address: DARBY & DARBY P.C., P.O. BOX 770, Church Street Station, New York, NY, 10008-0770, US

(73) Assignee: ADITECH PHARMA AB, Malmoe (SE)

(21) Appl. No.: 11/994,302

(22) PCT Filed: Jul. 7, 2006
(86) PCT No.: PCT/DK06/00402

§ 371 Date: Dec. 28, 2007

(30) Foreign Application Priority Data
Jul. 7, 2005 (DK) PA200501004

(51) Int. Cl. A61K 31/401; A61K 31/225; C07C 69/34; A61P 17/00; A61P 17/06; C07D 207/16

(52) U.S. Cl. 514/423; 560/190; 514/547; 548/535

(57) ABSTRACT

The present invention relates to novel amino acid salts of fumaric acid monoalkylesters. The salts are suitable for use as active substances in the treatment of e.g. psoriasis or other hyperproliferative, inflammatory or autoimmune disorders.

### FIELD OF THE INVENTION

The present invention relates to novel amino acid salts of fumaric acid monoalkylesters. The salts are suitable for use as active substances in the treatment of e.g. psoriasis or other hyperproliferative, inflammatory or autoimmune disorders either alone or in combination with other pharmaceuticals such as e.g. another fumaric acid ester.

### BACKGROUND OF THE INVENTION

Fumaric acid esters, i.e. dimethylfumarate in combination with ethylhydrogenfumarate have been used in the treatment of psoriasis for many years. The combination is marketed under the tradename Fumaderm®. It is in the form of tablets intended for oral use and it is available in two different dosage strengths (Fumaderm® initial and Fumaderm®):

| | Fumaderm ® Initial | Fumaderm ® |
|---|---|---|
| Dimethylfumarate | 30 mg | 120 mg |
| Ethylhydrogenfumarate, | 67 mg | 87 mg |
| calcium salt | | |
| Ethylhydrogenfumarate, | 5 mg | 5 mg |
| Magnesium salt | | |
| Etylhydrogenfumarate, | 3 mg | 3 mg |
| Zinc salt | | |

The two strengths are intended to be applied in an individually based dose regimen starting with Fumaderm® initial in an escalating dose, and then after e.g. three weeks of treatment switching to Fumaderm®. Both Fumaderm® initial and Fumaderm® are enteric coated tablets.

Another marketed composition is Fumaraat 120® containing 120 mg of dimethylfumarate and 95 mg of calcium monoethylfumarate (TioFarma, Oud-Beijerland, Netherlands). In a recent publication (Litjens et al. Br. J. Clin. Pharmacol. 2004, vol. 58:4, pp. 429-432), the pharmacokinetic profile of Fumaraat 120® is described in healthy subjects. The results show that a single oral dose of Fumaraat 120® is followed by a rise in serum monomethylfumarate concentration and only negligible concentrations of dimethylfumarate and fumaric acid is observed. The results indicate that dimethylfumarate is rapidly hydrolyzed to monomethylfumarate in an alkaline environment, but according to the authors not in an acid environment. As the composition is enteric coated, it is contemplated that the uptake of fumarate takes place mainly in the small intestine, where dimethylfumarate before uptake is hydrolysed to the monoester due to an alkaline environment or it may rapidly be converted due to esterases in the circulation. Furthermore, the study shows that tₘₐₓ and cₘₐₓ are subject to food effect, i.e. tₘₐₓ is prolonged (mean for fasted conditions is 182 min, whereas for fed conditions mean is 361 min) [lag time is 90 min for fasted and 300 min for fed] and cₘₐₓ is decreased (fasted: 0.84 mg/l, fed: 0.48 mg/l) by concomitant food-intake. Another study (Reddingius W. G. Bioanalysis and Pharmacokinetics of Fumarates in Humans. Dissertation ETH Zurich No. 12199)1997) in healthy subjects with two tablets of Fumaderm® P forte revealed cₘₐₓ values (determined as monoethyl- or monomethylfumarate) in a range from 1.0 to 2.4 μg/ml and a tₘₐₓ in a range of from 4.8 to 6.0 hours.

U.S. Pat. No. 6,277,882 and U.S. Pat. No. 6,355,676 disclose respectively the use of alkyl hydrogen fumarates and the use of certain fumaric acid mono alkyl ester salts for preparing micro tablets for treating psoriasis, psoriatic arthritis, neurodermatitis and enteritis regionalis Crohn. U.S. Pat. No. 6,509,376 discloses the use of certain dialkyl fumarates for the preparation of pharmaceutical preparations for use in transplantation medicine or the therapy of autoimmune diseases in the form of micro tablets or pellets. U.S. Pat. No. 5,424,332 discloses calcium, magnesium, zinc and iron salts of fumaric acid monoalkyl esters. U.S. Pat. No. 6,359,003 discloses treatment of transplant rejection by selectively suppressing host-versus-graft reaction using monoalkyl fumarate or its salt. U.S. Pat. No. 4,959,389 disclose compositions containing different salts of fumaric acid monoalkyl ester alone or in combination with dialkyl fumarate. The Case report “Treatment of disseminated granuloma annulare with fumaric acid esters” from BMC Dermatology, vol. 2, no. 5, 2002, relates to treatment with fumaric acid esters. US 2004/0038889 discloses use of fumaric acid amides for the therapy of an autoimmune disease, mithchondrial diseases, and NF-kappaB mediated diseases and in transplantation medicine. WO 89/01830 discloses fumaric acid diamides and monoamides for treatment of psoriasis.

However, therapy with fumarates like e.g. Fumaderm® frequently gives rise to flushing and/or gastro-intestinal side effects such as e.g. fullness, diarrhea, upper abdominal cramps, flatulence and nausea.

Furthermore, the present commercially available product contains a combination of two different esters of which one of the esters (namely the ethylhydrogenfumarate which is the monoethylester of fumaric acid) is present in three different salt forms (i.e. the calcium, magnesium and zinc salt). Although each individual form may have its own therapeutic profile it would be advantageous to have a much simpler product, if possible, in order to obtain a suitable therapeutic effect.

Accordingly, there is a need to develop novel drug compounds of therapeutically or prophylactically active fumaric acid esters that provide an alternative and potentially improved treatment e.g. with a reduction in flushing and/or reduction in gastro-intestinal related side effects upon oral administration and/or increased bioavailability.

### SUMMARY OF THE INVENTION

The present invention provides in one aspect new amino acid salts of monoalkylesters of fumaric acid of the general formula (I)

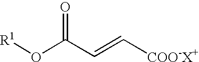

formula (I)

wherein
R¹ is C₁₋₅alkyl and
X⁺ is a protonated form of an amino acid, and any enantiomers or racemic mixtures thereof.

These novel drug compounds are contemplated to lead to an improved treatment of conditions susceptible to fumarate and/or fumaric acid ester treatment.

The mono- and dimethylester as well as the mono- and diethylester of fumaric acid have a poor solubility in water and this may be a factor leading to poor bioavailability (the bioavailability for the dimethylester of fumaric acid is regarded as very variable after oral administration). It is contemplated, that the salts according to the invention have the advantage that the amino acid part of the salt facilitates the absorption of the pharmaceutically active ingredient part of the salt in the intestine by the mechanisms that facilitate amino acid absorption, the so-called sodium co-transport and facilitated diffusion, possibly leading to an increased bioavailability.

Formation of the amino acid salts according to the invention may lead to a more suitable solubility in water or to a more suitable hydrophilic-lipophilic balance and, furthermore, due to the beneficial effect of the amino acid itself, the novel salts according to the invention are contemplated to lead to an improved treatment regimen.

In further aspects, the invention relates to a pharmaceutical composition comprising a compound according to the invention.

In further aspects, the invention provides methods of treatment and use of said new amino acid salts of monoalkylesters of fumaric acid in medicine and/or for combating tissue degenerative processes and/or more specifically in the treatment of conditions such as Psoriasis, Psoriatic arthritis, Neurodermatitis, atopic dermatitis, Inflammatory bowel disease, such as Crohn's disease and Ulcerative colitis, Autoimmune diseases such as Polyarthritis, Multiple sclerosis (MS), Juvenile-onset diabetes, Hashimoto's thyroiditis, Grave's disease, SLE (systemic lupus erythematosus), Sjögren's syndrome, Pernicious anemia, Chronic active (lupoid) hepatitis, rheumatoid arthritis (RA) and optic neuritis, pain such as radicular pain, pain associated with radiculopathy, neuropathic pain or sciatica/sciatic pain; or for treatment of any of the following conditions: prevention of rejection following organ transplantation; Sarcoidosis; Necrobiosis lipoidica; and/or Granuloma annulare, or for treatment of lupus nephritis, myasthenia gravis, uveitis, refractory uveitis, vernal conjunctivitis, pemphigus vulgaris, or scleroderma.

In another aspect of the invention, the use of said new amino acid salts of monoalkylesters of fumaric acid for the manufacture of a pharmaceutical composition is provided. In another further aspect, pharmaceutical compositions are provided. In yet further aspects, methods for preparation of such new salts are provided.

### DISCLOSURE OF THE INVENTION

The present invention provides in one aspect new amino acid salts of monoalkylesters of fumaric acid of the general formula (I)

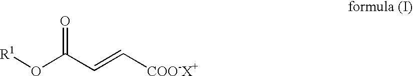

wherein
R¹ is C₁₋₅alkyl and
X⁺ is a protonated form of an amino acid, and any enantiomers or racemic mixtures thereof.

The compounds of the present invention may be chiral, and it is intended that any enantiomers, as separated, pure or partially purified enantiomers or racemic mixtures thereof are included within the scope of the invention.

In one aspect of the invention, the compound according to the invention is a D-enantiomer.

The present invention provides in a further aspect new amino acid salts of monoalkylesters of fumaric acid of the general formula (I)

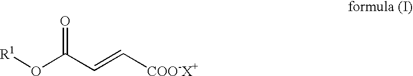

wherein
R¹ is C₁₋₅alkyl and
X⁺ is a protonated form of an amino acid.

Accordingly, the present invention relates to novel amino acid salts of a mono-(C₁₋₅)alkylester of fumaric acid that may be used alone or in combination treatment e.g. with a di-(C₁₋₅)alkylester of fumaric acid or other active substances.

The term “(C₁₋₅)alkyl” or “C₁₋₅alkyl” refers to a straight-chained or branched alkyl group having from one to five carbon atoms inclusive such as methyl, ethyl, 1-propyl, 2-propyl, isopropyl, 1-butyl, 2-butyl, 2-methyl-2-propyl, 2-methyl-1-propyl, or pentyl.

In a further aspect of the invention, R¹ is methyl or ethyl, preferably methyl.

The present invention also provides compositions including controlled release compositions comprising a novel salt according to the invention as well as to the use of the novel salts in medicine. Furthermore, the present invention provides a method for the manufacturing of the novel salts according to the invention.

In one aspect of the invention, a composition according to the invention comprising a novel salt may—upon oral administration and in comparison to that obtained after oral administration of Fumaderm® tablets in an equivalent dosage—give a reduction in GI (gastro-intestinal) related side-effects and/or reduce flushing (frequency and/or severity).

A suitable way of reducing the gastro-intestinal related side effects and/or flushing is likely to be by administration of a novel salt in the form of a controlled release composition.

As used in the present invention, a gastro-intestinal (GI) side effect may include, but is not limited to diarrhea, stomach ache, stomach pain, abdominal pain, abdominal cramps, nausea, flatulence, tenesmus, meteorism, an increased frequency of stools, a feeling of fullness and upper abdominal cramps.

In the present context, a reduction of GI related side effects is intended to denote a decrease in severity and/or incidence among a given treated patient population, compared to the GI side effects observed after administration of the composition according to the invention compared with that of Fumaderm®. A reduction in GI related side effects according to this definition could thus be construed as a substantial reduction in incidence of any of the GI side effect listed above, such as at least a 10% reduction in incidence or more preferably at least 20% reduction in incidence or even more preferably a more than 30% reduction in incidence. A reduction in GI related side effect can also be expressed as a substantial reduction in severity in any of the GI side effects listed above, such as a reduction in severity and/or frequency of diarrhea, stomach ache, stomach pain, abdominal pain, abdominal cramps, nausea, flatulence, tenesmus, meteorism, increased frequency of stools, a feeling of fullness or upper abdominal cramps. The reduction of GI related side effects, as described above, can be monitored in a clinical trial setting, either comparing the administration of the composition according to the invention head on with Fumaderm® or with placebo. In case of a placebo controlled trial, the incidence of GI related side effects in the patients receiving the composition according to the invention compared to the placebo group, can be compared to historical trials comparing Fumaderm® to placebo (see e.g. Altmeyer et al, J. Am. Acad. Dermatol. 1994; full reference: Altmeyer P J et al, Antipsoriatic effect of fumaric acid derivatives. Results of a multicenter double-blind study in 100 patients. J. Am. Acad. Dermatol. 1994; 30:977-81). Typically, patients suffering from psoriasis are included in such a study, and typically more than 10% of the body surface area will be affected by psoriasis (severe psoriasis). However, patients in whom between 2 and 10 percent of the body surface area is affected can also be included (moderate psoriasis). Patients can also be selected based on the psoriasis area severity index (PASI). Typically, patients within a certain range of PASI are included, such as between 10 and 40, or such as between 12 and 30, or such as between 15 and 25 or >10 or >12 or >16. Patients with any type of psoriasis may be included (chronic plaque type, exanthematic guttate type, pustular type, psoriatic erythroderma or palmoplantar type), but in some cases only patients with the chronic plaque type are included. About 15 to 20 patients in each treatment group (composition according to the invention and Fumaderm® or placebo) are sufficient in most cases, but more preferably about 30 to 50 patients are included in each arm of the study. Total study duration can be as short as one day to one week, but more preferably the study will run for 8 weeks to 12 weeks or up to 16 weeks. The side effects can e.g. be assessed as the total number of times a certain side effect was reported in each group (irrespective of how many patients have experienced the side effect), or the side effects can be assessed as the number of patients that have experienced a certain side effect a certain number of times, such as at least once or at least twice or at least three times during the duration of the study. Furthermore, the severity of a side effect can be monitored, or a certain severity of a side effect can be required for it to qualify as a side effect in the study. A convenient way of assessing the severity of a side effect is via a visual analogue (VAS) scale.

In the present context the term “flushing” describes episodic attacks of redness of the skin together with a sensation of warmth or burning of the face, neck, and less frequently the upper trunk and abdomen. It is the transient nature of the attacks that distinguishes flushing from the persistent erythema of photosensitivity or acute contact reactions. Repeated flushing over a prolonged period of time can lead to telangiectasia and occasionally to classical rosacea of the face (Greaves M W. Flushing and flushing syndromes, rosacea and perioral dermatitis. In: Champion R H, et al, eds. Rook/Wilkinson/Ebling textbook of dermatology, 6^{th} ed., vol. 3. Oxford, UK: Blackwell Scientific, 1998: 2099-2104).

In the present context, a reduction of flushing is intended to denote a decrease in severity and/or incidence/frequency among a given treated patient population of flushing observed after administration of the composition according to the invention compared with flushing observed after administration of Fumaderm® and can be measured e.g as described by O'toole et al. Cancer 2000, 88(4): p. 770-776. A reduction in flushing according to this definition could thus be construed as a substantial reduction in incidence or severity of flushing. In one aspect of the invention, the incidence of flushing is reduced by at least about a third, in another aspect of the invention the incidence is reduced by half, and in a further aspect of the invention, the flushing incidence is reduced by about two thirds or more. Likewise, the severity is in one aspect of the invention reduced by at least about a third, in another aspect of the invention by at least half, and in a further aspect of the invention by at least about two thirds. Clearly a one hundred percent reduction in flushing incidence and severity is most preferable, but is not required. The reduction of flushing, as described above, can be monitored in a clinical trial setting, e.g. comparing the administration of the compound according to the invention compared with treatment with e.g. administration of Fumaderm®. In case of a Fumaderm® controlled trial, the incidence and severity, defined as mild, moderate or severe, of flushing in the patients receiving the compound according to the invention compared to the Fumaderm® group, can be compared. Typically, patients suffering from psoriasis are included in such a study, and typically more than 10% of the body surface area will be affected by psoriasis (severe psoriasis). However, patients in whom between 2 and 10 percent of the body surface area is affected can also be included (moderate psoriasis). Patients can also be selected based on the psoriasis area severity index (PASI). Typically, patients within a certain range of PASI are included, such as between 10 and 40, or such as between 12 and 30, or such as between 15 and 25 or >10 or >12 or >16. Patients with any type of psoriasis may be included (chronic plaque type, exanthematic guttate type, pustular type, psoriatic erythroderma or palmoplantar type), but in some cases only patients with the chronic plaque type are included. About 15 to 20 patients in each treatment group are sufficient in most cases, but more preferably about 30 to 50 patients are included in each arm of the study. Total study duration can be as short as one day to one week, but more preferably the study will run for 8 weeks to 12 weeks or up to 16 weeks. The side effects can e.g. be assessed as the total number of times a certain side effect was reported in each group (irrespective of how many patients have experienced the side effect), or the side effects can be assessed as the number of patients that have experienced a certain side effect a certain number of times, such as at least once or at least twice or at least three times during the duration of the study. Furthermore, the severity of a side effect can be monitored, or a certain severity of a side effect can be required for it to qualify as a side effect in the study. A convenient way of assessing the severity of a side effect is via a visual analogue (VAS) scale.

Intestinal permeability of the compounds according to the invention may be determined using several different methods in the art. Intestinal permeability may be determined e.g. as described by Werdenberg et al. (BioPharm. Drug Dispos. 24: 259-273 (2003)) by isolated intestinal mucosa as well as by Caco 2 cell mono layers in order to obtain estimates of the fraction of the dose absorbed for these compounds (as also described in example 11).

Amino Acids

The term “amino acid” as used in the present context describes a group of molecules that contains both amino and carboxylic acid functional groups, and esters and amides thereof. The amino acids may be alpha or beta amino acids. The alpha amino acids are those amino acids in which the amino and carboxylate functionalities are attached to the same carbon atom and which may be represented by the general formula (a)

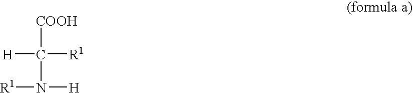

wherein R¹ is a side chain from either a naturally occurring or a modified or unusual alpha-amino acid and R² is hydrogen or C₁₋₅alkyl group.

The above configuration around the asymmetric-carbon atom constitutes the fundamental unit of so-called naturally occurring amino acids comprising alpha-amino acids such as glycine, alanine, valine, norvaline, isovaline, leucine, norleucine, isoleucine, methionine, phenylalanine, tryptophan, serine, threonine, cysteine, penicillamine, tyrosine, asparagine, glutamine, aspartic acid, glutamic acid, ornithine, lysine, arginine, histidine, proline, 4-hydroxy-proline, and pipecolic acid.

Except for glycine, where R=H, amino acids occur in two possible optical isomers, called “D” and “L”. L-amino acids represent the vast majority of amino acids found in proteins.

The term “amino acid” as used in the present context also includes so-called modified or unusual amino acids (in the following “modified amino acids”). Examples of such are, e.g., 2-aminoadipic acid, 3-aminoadipic acid, beta-alanine (or beta-aminopropionic acid), 2-aminobutyric acid, 4-aminobutyric acid or piperidinic acid, pipecolic acid, 6-aminocaproic acid, 2-aminoheptanoic acid, 2-aminoisobutyric acid, 3-aminoisobutyric acid, 2-aminopimelic acid, canavanine, 3-(3-carboxyphenyl)alanine, cystine, 2,4-diaminobutyric acid, desmosine, mimosine, 2,2-diaminopimelic acid, 2,3-diaminopropionic acid, N-ethylglycine, N-ethylasparagine, hydroxylysine, allo-hydroxylysine, 3-hydroxyproline, isodesmosine, allo-isoleucine, N-methylglycine (or sarcosine), 2-(methylenecyclopropyi)glycine, N-methylisoleucine, azaleucine, 2-amino-4-methylcaproic acid, 6-N-methyllysine, 4-methylglutamic acid, S-methylcysteine, S-(prop-1-enyl)cysteine and N-methylvaline

Examples of side chains (R¹ in above forumula) are hydrogen (glycine itself), deuterium (deuterated glycine), methyl(alanine), cyanomethyl(beta-cyano-alanin), ethyl, 1-propyl (norvaline), 2-propyl(valine), 2methyl-1-propyl(leucine), 2-hydroxy-2-methyl-1-propyl (beta-hydroxy-leucine), 1-butyl(norleucine), 2-butyl(isoleucine), methylthioethyl (methionine), benzyl(phenylalanine), p-amino-benzyl(p-amino-phenylalanine), p-iodo-benzyl (p-iodo-phenylalanine), p-fluoro-benzyl(p-fluoro-phenylalanine), p-bromo-benzyl(p-bromo-phenylalanine), p-chloro-benzyl(p-chloro-phenylalanine), p-nitro-benzyl(p-nitro-phenylalanine), 3-pyridylmethyl(beta-(3-pyridyl)-alanine), 3,5-diiodo-4-hydroxy-benzyl (3,5-diiodo-tyrosine), 3,5-dibromo-4-hydroxy-benzyl(3,5-dibromo-tyrosine), 3,5-dichloro-4-hydroxy-benzyl (3,5-dichloro-tyrosine), 3,5-difluoro-4-hydroxy-benzyl(3,5-difluoro-tyrosine), 4-methoxy-benzyl(O-methyl-tyrosin), 2-naphtylmethyl(beta-(2-naphtyl)-alanin), 1-naphtylmethyl(beta-(1-naphtyl)-alanin), 3-indolylmethyl(tryptophan), hydroxymethyl (serine), 1-hydroxyethyl(threonine), mercaptomethyl(cysteine), 2-mercapto-2-propyl (penicillamine), 4-hydroxybenzyl(tyrosine), aminocarbonylmethyl(asparagine), 2-aminocarbonylethyl (glutamine), carboxymethyl(aspartic acid), 2-carboxyethyl(glutamic acid), aminomethyl(α,β-diaminopropionic acid), 2-aminoethyl(alfa,gamma-diaminobutyric acid), 3-amino-propyl(ornithine), 4-amino-1-butyl(lysine), 3-guanidino-1-propyl(arginine), and 4-imidazolylmethyl(histidine), 1,3-propylene, 2-hydroxy-1,3-propylene, or 1,4-butylene forming a pyrrolidine ring, a 3-hydroxypyrrolidine ring, or a piperidine ring, respectively, involving the neighbouring carbon atom and a nitrogen atom (proline, 4-hydroxy-proline, and pipecolic acid, respectively).

The natural amino acids may be grouped into three major classes, according to their solubility in aqueous solution. The first class is comprised by amino acids that are non-polar and thus exhibit a relatively low solubility in water. The second class comprises amino acids that contain uncharged polar groups, while the third class contains a polar group that is charged. A further subdivision of the amino acids could be proposed, this including amino acids with sulphur atoms and those without. Only three amino acids, namely methionine, cystine and cysteine, contain sulphur and methionine belongs to the class of non-polar groups while cystine and cysteine belong to the class of polar uncharged groups.

In aqueous solution, the amino acids may act as zwitterions, that is, the amino acids are both acids and bases and the degree of protonation and de-protonation depends on the pH-value of the solution. At the biologically important conditions with pH-values close to 7, the carboxylic acid moiety is most frequently de-protonated and the amino group is protonated. In the present context the term “protonated” amino acid describes an amino acid where the hydrogen of one of the carboxylic acids from the fumaric acid resides on the —NH₂ of the amino acid.

The amino acid salts according to the invention are thus formed between an amino acid and a C₁₋₅alkyl ester of fumaric acid by ionic interactions of the amino group of the amino acid and the carboxylic acid group of the ester thus forming a coordination compound of formula I. The bond is thus of ionic type with two charged species. The bonds can be broken by contact with polar solutes such as water, alcohol or glacial acetic acid. In highly polar solutes such as water, the solubility of the salt as well as the degree of dissociation is expected to be high with, however, a maximum level that depends on the type of amino acid, on temperature and on the pH value. In general, equilibrium exists between the neutral undissociated species that are partly dissociated into charged ionic species, as illustrated in below reaction equation, where glycine coordinates to the acid moiety of MMF:

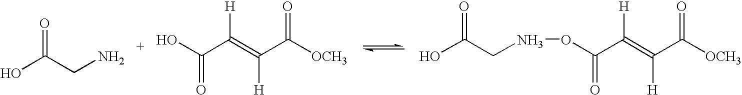

There exists an equilibrium of protonated and deprotonated species, as follows:

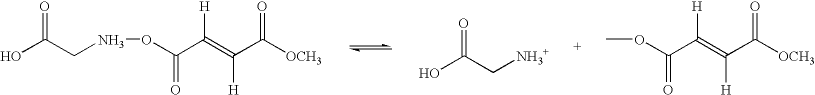

According to this reaction, the equilibrium may be displaced towards the left-hand side of undissociated species by adding a surplus of protonated amino acids, such as the hydro-chloride, or by adding fumarates, such as sodium fumarate. Under these conditions, it is contemplated that the uptake and transfer across membranes is enhanced by promoting conditions that favour the stability of the undissociated molecules. The coordination compounds of amino acids and e.g. MMF are expected to be highly soluble in water because of the high solubility of amino acids. However, in less polar solvents, the degree of dissociation may not be predominant and in non-polar environment, the molecule remains undissociated.

In one aspect of the invention, the amino acid is selected from the group consisting of natural amino acids such as glycine, serine, valine, histidine, threonine, leucine, isoleucine, cysteine, methionine, phenylalanine, tyrosine, proline, tryptophan, aspartic acid, glutamic acid, lysine, arginine, alanine, asparagine, glutamine, and ornithine. In a further aspect of the invention, the amino acid is selected from the group consisting of lysine, arginine, glutamine, histidine, ornithine and tryptophan. In still a further aspect of the invention, the amino acid is lysine

Active Substance

In an aspect of the invention, the fumaric acid ester is a mono-(C₁₋₅)alkylester of fumaric acid that is present in the form of an amino acid salt according to general formula I.

In a further aspect of the invention, the compounds according to the invention is selected from the group consisting of
amino acid salts of monomethylester of fumaric acid,
amino acid salts of monoethylester of fumaric acid,
amino acid salts of monopropylester of fumaric acid,
amino acid salts of monobutylester of fumaric acid, and
amino acid salts of monopentylester of fumaric acid.

In yet a further aspect of the invention, the compound is an amino acid salt of the monomethylester of fumaric acid.

In a further aspect of the invention, the compound according to the invention is selected from the group consisting of:
(S)-2-hydro-2,6-diaminohexanal-(E)-methoxy-4-oxobut-2-enoate(lysine monomethylfumarate),
(S)-6-hydro-2,6-diaminohexanal-(E)-methoxy-4-oxobut-2-enoate(lysine monomethylfumarate),
2-hydro-amino-((E)-methoxy-4-oxobut-2-enoate)-3-hydroxybutanoic acid (threonine monomethylfumarate),
hydro-pyrrolidine-((E)-methoxy-4-oxobut-2-enoate)-2-carboxylic acid (proline monomethylfumarate),
(S)-2-hydro-amino-((E)-methoxy-4-oxobut-2-enoate)-3-(1H-imidazol-5-yl)propanoic acid (histidine monomethylfumarate),
2-hydro-((E)-methoxy-4-oxobut-2-enoate)-aminopropanoic acid (alanine monomethylfumarate),
2-hydro-amino-((E)-methoxy-4-oxobut-2-enoate)-acetic acid (glycine monomethylfumarate),
2-hydro-amino-((E)-methoxy-4-oxobut-2-enoate)-3-hydroxypropanoic acid (serine monomethylfumarate),
2-hydro-amino-((E)-methoxy-4-oxobut-2-enoate)-5-guanidinopentanoic acid (arginine monomethylfumarate),
2-hydro-amino-((E)-methoxy-4-oxobut-2-enoate)-3-mercaptopropanoic acid (cystein monomethylfumarate),
2-hydro-2,4-diamino-((E)-methoxy-4-oxobut-2-enoate)-4-oxobutanoic acid (asparagine monomethylfumarate) and
4-hydro-2,4-diamino-((E)-methoxy-4-oxobut-2-enoate)-4-oxobutanoic acid (asparagine monomethylfumarate).

In a further aspect of the invention, the compound according to the invention is a lysine salt of the monomethylester of fumaric acid.

In one aspect of the invention, the active substance in a composition of the invention is an amino acid salt of a mono(C₁₋₅)alkylester of fumaric acid such as monomethylfumarate, monoethylfumarate, monopropylfumarate, monobutylfumarate and monopentylfumarate.

The active substance may be used in combination with another fumaric acid ester such as a dialkylfumarate like e.g. dimethylfumarate, diethylfumarate, dipropylfumarate, dibutylfumarate, dipentylfumarate, methyl-ethylfumarate, methyl-propylfumarate, methyl-butylfumarate or methyl-pentylfumarate, or monoalkylfumarates such as monomethylfumarate, monoethylfumarate, monopropylfumarate, monobutylfumarate or monopentylfumarate including pharmaceutically acceptable salts thereof.

In another aspect, a composition according to the invention comprises an amino acid salt of a mono(C₁₋₅)alkylester of fumaric acid together with a di(C₁₋₅)alkylester of fumaric acid (e.g. dimethylfumarate) as the active substances.

In a further aspect, the composition according to the invention comprises as active substances a combination of an amino acid salt of a mono(C₁₋₅)alkylester of fumaric acid and a mono(C₁₋₅)alkylester of fumaric acid (e.g. monomethylfumarate) optionally in the form of a pharmaceutically acceptable salt like e.g. its sodium, potassium, strontium, calcium, magnesium and/or zinc salt.

Synthesis of Amino Acid Salts of Fumaric Acid Monoesters According to the Invention

Fumaric acid, its monomethyl ester and its dimethylester are well known compounds that may be isolated from plants or synthesized (K. S. Rao and S. H. Mishra, J. Ethnopharmacology, vol. 60 (3), a998, pp. 207-213). The synthesis of the monomethyl ester of fumaric acid is not necessarily straightforward because of symmetry. Accordingly, attempts to synthesize the monomethyl ester by adding methanol to fumaric acid may inevitably lead to formation of the dimethyl ester. In addition, the synthesis may be complicated by the presence of the double bond, which under elevated temperature and pressure may hydrolyse and produce oxalic acid. The monomethyl fumarate may be synthesised by hydrolysis of methyl hydrogen fumarate following the method by Spatz and Stone (J. Org. Chem., vol. 23 (10), 1958, pp. 1559-1560).

Several ways of producing the amino acid salts of fumaric acid monoalkylesters according to the invention may be contemplated.

In an aspect of the invention, a method for preparing an amino acid salt according to the invention is provided, comprising formation of the hydroalkylfumarate of e.g. lysine, according to the procedure for production of lysine hydroacetate described by M. Schnabelrauch, S. Wittmann, K. Rahn, U. Möllmann, R. Reissbrodt and L. Heinisch, BioMetals, 13 (2000) pp. 333-348. In another aspect of the invention, the procedure described by A. Buononato in U.S. Pat. No. 6,730,693 B2 may be followed. Most amino acids form the hydrochloride upon precipitation with hydrochloric acid and, similarly, hydroacetates are synthesized by precipitation in solutions containing acetic acid. Salts of amino acids, lysine in particular, and e.g. ibuprofen (or acetylsalicylate) may be synthesized by precipitation from solvents of ethanol-water mixtures followed by evaporation of the solvent (L Baydoun, A. Düvel, R. Daniels, A. Drust, T. Goldhagen, I. Schwan, C. Zeidler and C. C. Müller-Goymann, Proc. Jahrestagung der DPhG, Würzburg, Aug. 11, 2003).

Dosage

Apart from providing pharmaceutical compositions having different content of the compounds according to the invention present, the invention in one aspect also provides kits containing two or more containers e.g. with compositions having various amounts of the compounds according to the invention included. Such kits are e.g suitable for use in those situations where an increasing dosage is required over time.

In one aspect of the invention, an up-scale of the dosage is e.g. ½ dose for 3-7 days, such as 7 days, thereafter full dose, alternatively ⅓ of the dose for 3-7 days such as 7 days, thereafter ⅔ of the dose for 3-7 days such as 7 days, thereafter full dose, alternatively full dose from day one.

In one aspect of the invention, a pharmaceutical composition wherein the amount of compound according to the invention in a dosage form is from 90 mg to 1000 mg active substance, such as 90 mg to 600 mg active substance, such as 90 mg to 540 mg active substance, such as 90 mg to 500 mg active substance, such as 90 mg to 360 mg active substance, such as 90, 120, 180, 240, 360, 450, 480, 500, 540, 600 or 1000 mg active substance, is provided. In a further aspect of the invention the amount of active substance is 120, 180 or 240 mg active substance. In yet a further aspect of the invention, the amount of active substance is 180 or 360 mg.

The daily dosage of the pharmaceutical composition according to the invention that is administered to treat a patient depends on a number of factors among which are included, without limitation, weight and age and the underlying causes of the condition or disease to be treated, and is within the skill of a physician to determine. In one aspect of the invention the daily dosage can be e.g. from 240 to 360 mg active substance given in one to three doses, in another aspect from 360 to 480 mg active substance given in one to three doses, in another aspect 480 to 600 mg active substance given in one to three doses, in another aspect 600 to 720 mg active substance given in one to three doses, in another aspect 720 to 840 mg active substance given in one to three doses, in another aspect 840 to 960 mg active substance given in one to three doses and in yet another aspect 960 to 1080 mg active substance given in one to three doses.

In another aspect of the invention, a pharmaceutical composition in the form of a tablet is provided, such as a tablet which has a shape that makes it easy and convenient for a patient to swallow e.g. a tablet which has a rounded or a rod-like shape without any sharp edges.

In another aspect of the invention, a pharmaceutical composition in the form of a tablet designed to be divided into two or more parts, is provided.

The compositions according to the invention may be administered together with a meal or in relation to a meal such as e.g. in a time period corresponding to a range from at least about 30 minutes before a meal to about 2 hours after the meal, or the composition may be administered at any specific point(s) in time during the day.

In one embodiment, the total daily dose is given at bedtime, such as up to or about 30 minutes before bedtime, up to or about 60 minutes before bedtime, up to or about 90 minutes before bedtime, up to or about 120 minutes before bedtime or up to or about 180 minutes before bedtime.

In one aspect of the invention, the dosage of a compound according to the invention to be administered should provide a peak plasma concentration (Cₘₐₓ) of the corresponding alkyl fumarate in a range of from about 0.4 to about 4.0 mg l⁻¹ after a single dose administration to humans, such as from about 0.5 to about 3.0 mg l⁻¹ after a single dose administration to humans, such as from about 1.0 to about 2.5 mg l⁻¹ after a single dose administration to humans, such as from about 1.0 to about 2.0 mg l⁻¹ after a single dose administration to humans.

In another aspect of the invention, the dosage of a compound according to the invention to be administered should provide an area under the plasma concentration vs. time profile (AUC_{0-∞}) of the corresponding alkyl fumarate of from about 30 to 750, such as from about 30 to 600, from about 30 to 450, from about 30 to 300 or from about 30 to 150 mg×min l⁻¹ after a single dose administration to humans.

In another aspect of the invention, the total daily dosage of a compound according to the invention to be used should provide a clinical effect as measured by the percentage of subjects achieving a PASI 75 (a PASI reduction of ≧75% from baseline PASI) after 12 weeks of treatment of at least 20%, such as at least 30%, such as at least 40%, such as at least 50%, such as at least 60%, such as about 40%, such as about 50%.

In another aspect of the invention, the total daily dosage of a compound according to the invention to be used should provide a clinical effect as measured by the percentage of subjects achieving a PASI 75 (a PASI reduction of ≧75% from baseline PASI) after 16 weeks of treatment of at least 20%, such as at least 30%, such as at least 40%, such as at least 50%, such as at least 60%, such as about 40%, such as about 50%.

In another aspect of the invention, the total daily dosage of a compound according to the invention to be used should provide a clinical effect as measured by the percentage of subjects achieving a PASI 75 (a PASI reduction of ≧75% from baseline PASI) after 24 weeks of treatment of at least 30%, such as at least 40%, such as at least 50%, such as at least 60%, such as at least 70%, such as about 40%, such as about 50%, such as about 60%.

The clinical effect of the compounds according to the invention may be measured in a double-blind, placebo controlled, parallel-group study. Eligible patients for testing for the effect on e.g. psoriasis are e.g. patients who have had psoriasis (chronic, exanthematic guttate, erythrodermic, plamoplantar, or pustular) for at least 1 year. Patients should typically have a baseline PASI of 16-24 or ≧10, or ≧12. Systemic treatment should be discontinued 4 weeks before study initiation. Topical treatment should be discontinued 2 week before study initiation. Only topical salicylic acids and emollients should be allowed during the study period.

Patients should be randomised to either the placebo-group or to a group receiving the pharmaceutical composition according to the invention. The total number of patients to be included will depend on the specific study-design but may be e.g. 80 patients with 40 patients on placebo and 40 patients on active treatment.

The treatment period is 12-16 weeks or up to 24 weeks, or up to 52 weeks. The primary measure of efficacy is the reduction in PASI score between baseline and at the end of treatment, or the percentage of subjects achieving e.g. a PASI 75 (≧75% reduction from baseline in their PASI scores) or a PASI 90 (≧90% reduction from baseline in their PASI scores), or by determining the change in the physician's global assessment (PGA).

In one aspect of the invention, the compounds according to the invention have an increased dissolution rate compared to the calcium salt of monomethyl fumarate. In a further aspect of the invention, the compounds according to the invention have an increased dissolution rate leading to increased bioavailability compared to administration of monomethyl fumarate. In another aspect of the invention, the compounds according to the invention have an increased bioavailability in-vivo as compared to the administration of monomethyl fumarate.

Uses

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

The terms “disease”, “condition” and “disorder” as used herein are used interchangeably to specify a state of a patient which is not the normal physiological state of man.

The compounds, compositions and kits according to the invention are contemplated to be suitable for use in medicine and/or for combating tissue degenerative processes, hyperproliferative, inflammatory and/or autoimmune disorders and more specifically for the treatment of one or more of the following conditions:
a. Psoriasis
b. Psoriatic arthritis
c. Neurodermatitis, atopic dermatitis
d. Inflammatory bowel disease, such as
   i. Crohn's disease
   ii. Ulcerative colitis
e. Autoimmune diseases:
   i. Polyarthritis
   ii. Multiple sclerosis (MS)
   iii. Juvenile-onset diabetes mellitus
   iv. Hashimoto's thyroiditis
   v. Grave's disease
   vi. SLE (systemic lupus erythematosus)
   vii. Sjögren's syndrome
   viii. Pernicious anemia
   ix. Chronic active (lupoid) hepatitis
   x. Rheumatoid arthritis (RA)
   xi. Optic neuritis
f. Pain such as radicular pain, pain associated with radiculopathy, neuropathic pain or sciatica/sciatic pain
g. Organ transplantation (prevention of rejection)
h. Sarcoidosis
i. Necrobiosis lipoidica
j. Granuloma annulare

Moreover, the compounds, compositions and kits according to the invention may be used in the treatment of one or more of the following conditions lupus nephritis, myasthenia gravis, uveitis, refractory uveitis, vernal conjunctivitis, pemphigus vulgaris, and/or scleroderma.

Psoriasis has been proposed to potentially be associated with Crohn's disease (Najarian D J, Gottlieb A B, Connections between psoriasis and Crohn's disease. J Am Acad Dermatol. June 2003;48(6):805-21), celiac disease (Ojetti V et al, High prevalence of celiac disease in psoriasis. Am J Gastroenterol. November 2003;98(11):2574-5.), psychiatric or psychological disease, such as depression or a life crisis (Gupta M A, Gupta A K, Psychiatric and psychological co-morbidity in patients with dermatologic disorders: epidemiology and management. Am J Clin Dermatol. 2003;4(12):833-42. and Mallbris L et al, Psoriasis phenotype at disease onset: clinical characterization of 400 adult cases. J Invest Dermatol. March 2005;124(3):499-504.), overweight, diabetes mellitus, excess consumption of alcohol/alcoholism, as well as psoriatic arthritis.

The present invention thus relates in one aspect to a method of treating one or more conditions selected from the group consisting of psoriasis, psoriatic arthritis, neurodermatitis, inflammatory bowel disease, such as Crohn's disease and ulcerative colitis, autoimmune diseases such as polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), and optic neuritis, pain such as radicular pain, pain associated with radiculopathy, neuropathic pain or sciatica/sciatic pain, organ transplantation (prevention of rejection), sarcoidosis, necrobiosis lipoidica, granuloma annulare, lupus nephritis, myasthenia gravis, uveitis, refractory uveitis, vernal conjunctivitis, pemphigus vulgaris, and scleroderma, which method comprises administering orally to a patient in need thereof, an effective dosage of a of a compound according the invention.

The present invention relates in another aspect to the use of a compound according to the invention for the preparation of a medicament for the treatment of one or more conditions selected from the group consisting of psoriasis, psoriatic arthritis, neurodermatitis, inflammatory bowel disease, such as Crohn's disease and ulcerative colitis, autoimmune diseases such as polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), and optic neuritis, pain such as radicular pain, pain associated with radiculopathy, neuropathic pain or sciatica/sciatic pain, organ transplantation (prevention of rejection), sarcoidosis, necrobiosis lipoidica, granuloma annulare, lupus nephritis, myasthenia gravis, uveitis, refractory uveitis, vernal conjunctivitis, pemphigus vulgaris, and scleroderma.

In one aspect of the invention, a compound according to the invention for use in the treatment of one or more conditions, where the condition is selected from psoriasis, psoriatic arthritis, neurodermatitis and multiple sclerosis (MS), is provided. In yet a further aspect of the invention, a compound according to the invention for use in the treatment of psoriasis, is provided. In another aspect of the invention, a compound according to the invention for use in the treatment of psoriatic arthritis, is provided.

In yet a further aspect of the invention, a compound according to the invention for use in the treatment of multiple sclerosis, is provided.

Furthermore, the invention also relates to treating an individual suffering from one of the conditions in the abovementioned lists, more specifically multiple sclerosis, with a compound, composition or kit according to the invention, said individual further being in treatment with one or several compounds selected from the group consisting of PDB-0921 (P-005063, P-005088, P-005291, PDB-5613, PDB-5792), BGC-20-884, atorvastatin, Abatacept, alemtuzumab, Sativex, daclizumab, glatiramer acetate, ibudilast, interferon (Serono (\b1a)), interferon (AW (\a)), interferon (Biogen (\b1a)), interferon (Novartis (\b1b)), interferon (Hemispherx), alefacept, levetiracetam, memantine hydrochloride, mitoxantrone, rituximab, simvastatin, baclofen (intrathecal), Cannabis (SIMM-18), Corticotropin, MLN-3897, MLN-519 (LDP-519, PN-05, PS-519), AEG-35156 (AEG-161, AEG-35169, GEM-640), RG-2077 (CTLA4-Ig, RG-1059), TBC-4746, MMP-12 inhibitors (Serono), R-1295, TRX-1, CDP-323, SC-12267, MDX-1100, ACE inhibitors (GenoMed), Cannabinor (PRS-211375), AVE-9897, JNK inhibitors (Serono), TV-3606, MLN-3701, rHDL (ZLB, CSL), AGT-1, NeuroVax (AI-208, BV-13S1, BV-5S2, BV-6S5, IR-208), fontolizumab, atiprimod dimaleate (Symadex), IP-751 (ajulemic acid, CT-3, DMH-11C), IDN-6556, Talampanel (GYKI-53773, LY-293606, LY-300164), GPI-1485 (GPI-1005, GPI-1046, GPI-1152, GPI-1216), talotrexin ammonium, AVR-118, Onercept, merimepodib, ABT-874, laquinimod, APT-070C, interferon (Pepgen (tau), Tauferon), IL-18BP (Yeda), ISIS-107248 (ATL-1102), delmitide acetate, SGN-30, MM-093 (ABI.001), AMG-487 (CCX-395, T-487), Monarsen (EN-101), EMZ-701, INO-1001, chaperonin-10 (CBio, Cpn-10), INCB-003284, STA-5326, Tovaxin, MLN-1202, BHT-3009, c-6448, Aimspro, RPI-78M, JM-002, Peptide T (Advanced Immuni T), TV-5010, N-palmitoylethanolamide (Stief), E3 (Effective Pharmaceuticals), 808-2, FAR-404, MCT-215, MK-0812, GEM-SP, Pixantrone (BBR-2778), Dexanabinol (HU-211, PRS-211092, PRS-211095, PRS-211220), fingolimod hydrochloride (FTY-720, FTY-720A, FTY-726), fampridine-SR, pirfenidone, Theralux, temsirolimus, E-2007, teriflunomide, MBP-8298, interferon (Rentschler (\b)-2), CNTO-1275, cladribine (IVAX), HumaT4 (anti-CD4 MAb, Intracel), MV-57471, glial growth factor-2 (CeNeS), M1 MAbs (Acorda), neural stem cells (StemCells), stem cells (hESCs, Geron), CXCR3 antagonists (Pharmacopeia), SCS technology, Pharmaprojects No. 5480, Immunosuppressants (p53-69, GPC), NBI-59159, E-2050 (ER-129002-02), neuregulin-2 (Acorda), soluble CD8 (MiDex, Avidex), IBD gene therapy (AMT), DN-1921 (Dantes), VLA-4 antagonists (Uriach), MS therapy (sodium channel blocker, Genopia), Erythropoietin (WP-170, Warren), heparanase inhibitors (Progen), CD-200Fc, MS therapy (MHC inhibitors, Provid), SGN-35, Neliximab, SYN-5001, interferon (Syntonix (\b)), PP-0102, LOR-S03, CCX-634, TMC-2003, MRK-167 (CMPD-167), TNF-\a inhibitors (Xencor), ReaDex, PLX-647, inflamm/autoimmune ther (Mann), SPR-1401, Antidepressants (ND-1251, ND-1510, Neuro3d), PXS-64 (PXS-25), PXS-2000, AT-008, autoimmune disease ther (Alnyl), interferon (Nautilus (\b)), CO-14, hedgehog agonists (neurological), anti-IL-23 (Archemix), BGC-20-0134, MORAb-022, MIF inhibitor (Genzyme), INCB-3344, immune regulating hormones (Hol), NNZ-2566, NNZ-4921, RX-111 (BL-1030), CLT-001, BKT-104, PEG-IFN-\b (Enzon), AZD-5904, interferon (Bolder (\b)), CB-2 agonists, BTG, Kv1.3 channel blockers (4SC), PS-375179, CCX-915, vitamin D signal amplifiers-5, Scleroneurin, IGIV (Hemosol), inflamm/autoimmune ther (Apollo), QR-442, leupeptin+taurine (C-201, Neurodur, CepTor), anti-CD3 antibody (Diversa), MLN-0415, Rob-895, AZD-8797, CHR-1103, multiple sclerosis ther (Brain), interferon (Vakzine (\b)), CCR2 antagonists (Merck & Co), GEMS-001, Natalizumab, BG-12 (Panaclar), and mitoxantrone.

In another embodiment, the invention relates to treating an individual suffering from one of the conditions in the abovementioned lists, more specifically multiple sclerosis, with a compound, composition or kit according to the invention, said individual further being in treatment with one or several compounds selected from the group consisting of beta-interferon 1a, beta-interferon 1b, natalizumab, BG-12, glatiramer acetate, mitoxantrone and fingolimod hydrochloride (FTY-720, FTY-720A, FTY-726).

Furthermore, the invention also relates to treating an individual suffering from one of the conditions in the abovementioned lists, more specifically psoriasis or psoriatic arthritis, with a compound, composition or kit according to the invention, said individual further being in treatment with
a) a topical anti-psoriatic drug such as 1) vitamin D or derivatives thereof (calcipotriol, calcipotriene), 2) a corticosteroid (such as e.g. betamethasone, desoximethasone, fluocinolone, momethasone, hydrocortisone aceponate, fluticasone, clobethasol, clobethasone, hydrocortisone butyrate, desonide, triamcinolone or hydrocortisone), 3) tazaroten, 4) ditranol, 5) tacrolimus (FK-506) and other calcineurin inhibitors, such as pimecrolimus or 6) any combination of 1-5 and/or
b) an oral anti-psoriatic drug such as 1) an oral retinoid (such as acitretin or etretinate) combined or not combined with PUVA, 2) cyclosporine and other calcineurin inhibitors, such as ISA247, tacrolimus and pimecrolimus, 3) methotrexate, 4) hydroxyurea, 5) azathioprine, 6) sulphasalazine, 7) a fumarate derivative (such as e.g. Fumaderm or BG-12), 8) rosiglitazone (Avandia) and other peroxisome proliferator-activated-γ(PPARγ) agonists or modulators, such as pioglitazone, farglitazar, GW1929, GW7845, MC-555, MBX-102/MBX-10, MBX-1828, MBX-2044, CLX-0921, R-483, reglitazar, naveglitazar (LY-519818/LY-818), netoglitazone (MCC-555), CS-7017, troglitazone, ciglitazone, tesaglitazar, isaglitazone, balaglitazone, muraglitazar, TAK-654, LBM642, DRF 4158, EML 4156, T-174, TY-51501, TY-12780, VDO-52 or AMG-131(T131) or any combination of 1-8 and/or
c) a parenterally administered anti-psoriatic drug such as 1) alefacept (Amevive), 2) etanercept (Enbrel), 3) efalizumab (Raptiva), 4) onercept, 5) adalimumab (Humira) or any combination of 1-5 and/or
d) an inhibitor of TNF-α not mentioned in the list under section c) above (e.g. CDP 870 or infliximab (Remicade)), administered via an enteral or parenteral route and/or
e) tisocalicitrate and/or NCX 1022 and/or IDEC-131 and/or MEDI-507, and/or
f) An NSAID or a COX or a LOX inhibitor such as e.g. a COX-2 inhibitor or a COX/5-LOX inhibitor, and/or
g) an anti-diabetic or anti-obesity drug, such as biguanides such as metformin; metformin XR; a sulphonylurea such as chlorpropamide, glipizide, gliclazide, glyburide/glibenclamide or glimepiride; Glucovance (metformin+glyburide); Metaglip (glipizide+metformin); a peroxisome proliferator-activated-γ(PPARγ) agonist or modulator, such as rosiglitazone (Avandia), pioglitazone, farglitazar, GW1929, GW7845, MC-555, MBX-102/MBX-10, MBX-1828, MBX-2044, CLX-0921, R-483, reglitazar, naveglitazar (LY-519818/LY-818), netoglitazone (MCC-555), CS-7017, troglitazone, ciglitazone, tesaglitazar, isaglitazone, balaglitazone, muraglitazar, TAK-654, LBM642, DRF 4158, EML 4156, T-174, TY-51501, TY-12780, VDO-52 or AMG-131(T131); Avandamet (rosiglitazone+metformin); Actos (pioglitazone+metformin); Avandaryl(rosiglitazone maleate+glimepiride); a benzoimidazole such as FK-614; CS-917; TA-1095; ONO-5129; TAK-559; TAK-677/AJ-9667; a d-phenylalanine inducer such as senaglinide; c-3347; NBI-6024; ingliforib; BVT 3498; LY 929; SGLT2 inhibitors; CS 011; BIM 51077; R1438; R1439; R1440; R1498; R1499; AVE 0847; AVE 2268; AVE 5688; AVE 8134; TA-6666; AZD 6370; SSR 162369; TLK-17411; NN 2501; MK 431; KGA-2727; MK-767; CS-872; a beta-3 receptor antagonist such as N-5984; an alpha-glucosidase inhibitor such as acarbose, voglibose or miglitol; a glinitide/meglitinide analogue or carbamoylmethylbensoeic acid derivative such as mitiglinide, repaglinide or nateglinide; a DPP-IV inhibitor such as LAF 237 (vildagliptin), DPP728, P93/01, P32/98, PT-630 or saxagliptin; GLP-1 or GLP-1 analogues, such as exenatide, Exenatide-LAR, liraglutide (NN 2211), ZP 10/AVE 0010, LY 307161, betatropin, CJC-1131, GTP-010, SUN E7001 or AZM 134; pramlinitide acetate; insulin or insulin analogues, such as Humalog (insulin lispro), Humulin, Novolin, Novolog/NovoRapid (insulin aspart), Apidra (insulin glulisine), Lantus (insulin glargine), Exubera, Levemir/NN 304 (insulin detemir), AERx/NN 1998, Insuman, Pulmonary insulin or NN 344; sibutramine or other blockers of the presynaptic reuptake of serotonin and noradrenalin; orlistat and other inhibitors of GI lipases; β3-adrenergic receptor agonists; uncoupling proteins; (specific) antagonists of PPARγ (Peroxisome Proliferator-Activated Receptor γ); insulin secretagogues; rimonabant and other CB1 endocannabinoid receptor antagonists; bupropion; topiramate; leptin agonists; ciliary neurotrophic factor; peptide analogues of the human growth hormone fragment 177-191; cholecystokinin-A receptor agonists; melanocortin-3 agonists; noradrenergic drugs such as phentermine, diethylpropion, phendimetrazine or benzphetamine; or any combination of the anti-diabetic or anti-obesity drugs mentioned above, and/or

h) a drug potentially useful in the treatment of substance abuse e.g. alcohol abuse such as naltrexone, acamprosate, disulphiram or Vivitrex (naltrexone long acting injection), and/or,

i) a drug potentially useful in the treatment of Crohn's disease such as
1. 5-ASA compounds such as sulfasalazine, oral 5-ASA formulations or rectal 5-ASA formulations,
2. glucocorticosteroids such as systemic steroids (e.g. budesonide or prednisolone) or topically acting steroids (e.g. budesonide),
3. antibiotics such as metronidazole or quinolones (e.g. ciprofloxacine, ofloxacine, norfloxacine, levofloxacine or moxifloxacine),
4. immunosuppressives such as azathioprine, 6-mercaptopurine or methotrexate,
5. nutritional therapies such as elemental or polymeric formulas or pre- and probiotics,
6. biological therapies e.g. TNF-α inhibitors such as infliximab, adalimumab, CDP870, CDP571, etanercept or onercept,
7. symptomatic agents such as anti-diarrheals or anti-spasmodics.

Examples of suitable NSAIDs are piroxicam, diclofenac, nabumetone, propionic acids including naproxen, flurbiprofen, fenoprofen, ketoprofen and ibuprofen, fenamates including mefenamic acid, paracetamol, indomethacin, sulindac, meloxicam, apazone, pyrazolones including phenylbutazone, salicylates including aspirin.

Examples of suitable COX-2 inhibitors are rofecoxib (Vioxx), valdecoxib (Bextra), celecoxib (Celebrex), etoricoxib (Arcoxia), lumiracoxib (Prexige), parecoxib (Dynastat), deracoxib (Deram), tiracoxib, meloxicam, nimesolide, (1,1-dimethylheptyl)-6a,7,10,10a-tetrahydro-l-hydroxy-6,6dimethyl-6H-dibenzo[b,d]pyran carboxylic acid (CT-3), 2(5H)-Furanone, 5,5-dimethyl (l-methylethoxy)[4(methylsulfonyl)phenyl]-(DFP); Carprofen (RIMADYL), (Acetyloxy)-benzoic acid, 3-[(nitrooxy)methyllphenyl ester (NCX4016), P54 (CAS Reg. No. 130996 0) 2,6-Bis(1,1-dimethylethyl)[(E)-(2-ethyl-1,1-dioxoisothiazolidinylidene)methyl]phenoI (S-2474), 5(R)-Thio sulfonamide-3(2H)-benzofuranone (SVT-2016) and N-[3-(Fonnyl-amino)oxo phenoxy-4H benzopyran yl] methanesulfonamide (“T-614”); or a pharmaceutically acceptable salt thereof.

Examples of suitable COX/5-LOX inhibitors are licofelone (ML-3000 or [2,2-dimethyl-6-(4-chlorophenyl)-7-phenyl-2,3,dihydro-1H-pyrrolizine-5-yl]-acetic acid), di-tert-butylphenols, such as (E)-(5)-(3,5-di-tert-butyl-4-hydroxybenzylidence)-2-ethyl-1,2-isothiazolidine-1,1-dioxide (S-2474), darbufelone or tebufelone and pharmacologically active metabolites as well as derivatives such as dihydro-dimethyl-benzofuran and PGV-20229, dihydro-dimethyl-benzofuran, thiophene derived compounds such as RWJ-63556, N-hydroxy-N-methyl-4-(2,3-bis-(4-methoxyphenyl)-thiophen-5-yl)-butanamide (S19812), methoxytetrahydropyran derivatives, oxygenated xanthones such as 1,3,6,7-Tetrahydroxyxanthone (norathyriol)-pyrazole thiocarbamates, pyrazoles such as modified forms of phenidone containing compounds or the tri-flouro-benzole substituted pyrazoline derivative BW-755C, tepoxaline and derivatives and di-tert-butylpyrimidines.

It is contemplated that such combination therapy leads to an improved therapeutic response and/or an increased convenience for the individual, compared to said individual being treated without the compound, composition or kit according to the invention.

In a further aspect, the invention relates to a method of reducing side effects associated with oral treatment of any of the conditions a-j listed above, in which method the active pharmaceutical ingredient for treating said condition is used in combination with one or more of the following agents:
a) an antacid such as 1) magnesium hydroxide, 2) magnesium trisilicate, 3) aluminium hydroxyde gel, 3) sodium hydrogencarbonate, 4) magaldrat or any combination of 1-5 and/or
b) a histamine H-2 antagonist such as 1) cimetidine, 2) ranitidine, 3) nizatidine, 4) famotidine, 5) roxatidine, 6) lafutadine or any combination of 1-6 and/or
c) a cytoprotective agent such as 1) sucralfate, 2) tripotassium dictitratobismuthate, 3) carbenoxolone, 4) prostaglandin E-2 analogues such as misoprostol, 5) ecabet, 6) cetraxate HCl, 7) teprenone, 8) troxipide, 9) dicyclomine hydrochloride, 10) sofalcon or any combination of 1-10 and/or
d) a proton pump inhibitor (PPI) such as 1) omeprazole, 2) esomeprazole, 3) lansoproazole, 4) pantoprazole, 5) rabeprazole, 6) CS-526/R-105266, 7) AZD 0865, 8) soraprazan or any combination of 1-8.
e) an NSAID or a COX or a LOX inhibitor such as e.g. a COX-2 inhibitor or a COX/5-LOX inhibitor, and/or
f) pentoxifylline, e.g. at a dose range of from 400 to 800 mg/day.

In a specific aspect, the compound according to the invention is used in combination with a fumaric acid ester containing compound. In particular the fumaric acid ester containing compound is any and all of the salts contained in Fumaderm® or Fumaraat® or Panaclar® (BG-12) or described in U.S. Pat. No. 6,277,882, U.S. Pat. No. 6,355,676 or U.S. Pat. No. 6,509,376. The compound according to the invention may be provided in a formulation according to the present invention, or in any Fumaderm® or Fumaraat® or Panaclar® (BG-12) formulation or as e.g. described in U.S. Pat. No. 6,277,882, U.S. Pat. No. 6,355,676 U.S. Pat. No. 6,509,376, or PCT/DK2005/000648.

Cosmetic and/or Pharmaceutical Compositions

The novel salts of the invention may be presented in the form of a cosmetic or pharmaceutical composition. In a further aspect of the invention, the pharmaceutical composition is in the form of a controlled release composition. In one aspect of the invention, the pharmaceutical composition has an enteric coating.

The salts according to the invention may be used for preparing preparations for oral administration in the form of micro-pellets, micro-tablets, capsules (such as soft and hard gelatine capsules), granulates and tablets such as e.g. described in U.S. Pat. No. 6,509,376 or U.S. Pat. No. 6,355,676 incorporated herein by reference. Further suitable pharmaceutical preparations are preparations for cutaneous and transdermal administration in the form of ointments, plasters, lotions or shower preparations and for parenteral administration in the form of aqueous micro-dispersions, oil-in-water emulsions or oily solutions for rectal administration of suppositories or micro-enemas.

The novel salts may solve or reduce the problems related to the appearance of gastro-intestinal side-effects and/or flush side-effects upon oral administration of the known fumaric acid esters. Furthermore, by prolonging and/or delaying the release of the active substance from the composition it is envisaged that the local concentration of the active substance at specific sites of the gastrointestinal tract is reduced (compared with that of Fumaderm®) which in turn leads to a reduction in gastro-intestinal side-effects and/or flushing. Accordingly, compositions that enable a prolonged and/or a slow release of a compound according to the invention are within the scope of the present invention.

Such compositions are well-known to the skilled artisan and include e.g. diffusion-controlled drug delivery systems, osmotic pressure controlled drug delivery systems, erodible drug delivery systems etc. Moreover, there are pharmaceutical companies that based on a specific technology (such as mentioned above) can provide a specific composition with specific release characteristics of the active substance. Accordingly, a person skilled in the art will know how to obtain a suitable product once he has realized a specific need in respect of a particular drug substance. By way of example, Eurand is one of such companies that offer technical solutions in order to obtain a controlled release pharmaceutical composition containing a specific active substance and having specific requirements with respect to the release of the active substance from the composition (see e.g. http://www.eurand.com). Another company is MacroMed, Inc. that has developed a technology involving a so-called SQZgel™ (http://macromed.com, SQZgel™'S mechanism of action is a pH-sensitive polymer mixture combined with an outer coating. In the acidic environment of the stomach the polymer imbibes with water and swells, entrapping the drug. Upon entering the higher pH of the intestines, the polymer slowly shrinks, or “squeezes” at a “dialed-in” rate releasing the active composition in a sustained manner.), or Egalet a/s that has a specific extrusion based technology (http://www.egalet.com, Key elements of the Egalet® technology are a biodegradable coat and a matrix, comprising the active drug, which is surface erodible, hydrophobic and composed of PEG-stearate. One of the Egalet® technologies is the 2K Egalet® constant release system, which is a 2-component production model consisting of coat and matrix. The drug is evenly distributed throughout the Egalet® matrix for constant release over time). These and other technologies like e.g. the Eurand technologies Diffucaps (Drug release profiles are created by layering active drug onto a neutral core such as sugar spheres, crystals or granules followed by a rate-controlling, functional membrane. Diffucaps/Surecaps beads are small in size, approximately 1 mm or less in diameter. By incorporating beads of differing drug release profiles into hard gelatin capsules, combination release profiles can be achieved.), Diffutabs (The Diffutab technology incorporates a blend of hydrophilic polymers that control drug release through diffusion and erosion of a matrix tablet.), Minitabs (Eurand Minitabs are tiny (2 mm×2 mm) tablets containing gel-forming excipients that control drug release rate. Additional membranes may be added to further control release rate.), Orbexa (This technology produces beads that are of controlled size and density with a defined-based granulation extrusion and spheronization techniques. The resultant beads can be coated with release rate controlling membranes for additional release rate control and may be filled into capsules or provided in sachet form.) and SDS (Eurand's SDS technology uses functional polymers or a combination of functional polymers and specific additives, such as composite polymeric materials, to deliver a drug to a site of optimal absorption along the intestinal tract. In order to achieve this, Eurand first produces multiparticulate dosage forms such as Diffucaps or Eurand Minitabs, which incorporate the active drug. These dosage forms are then coated with pH dependent/independent polymeric membranes that will deliver the drug to the desired site. These are then filled into hard gelatin capsules.) are also of interest in the present context.

An interesting technology for use in formulating compositions according to the present invention is the so-called MeltDose® technology as described in WO 03/004001 (see http://www.lifecyclepharma.com. MeltDose® involves formulating solubilized, individual molecules into tablets. By formulating individual molecules, the primary limitation of oral absorption of drugs with low water-solubility is removed, and a superior bioavailability can be attained.). By employing this technology it is possible to obtain a particulate material that is suitable for processing into various pharmaceutical dosage forms e.g. in the form of pellets or tablets. Furthermore, the technology is suitable for use as it is possible to obtain a suitable release profile of the active substance, e.g. such as those release profiles described herein. In one embodiment, pellets suitable for use may have a mean particle size larger than 2000 μm. In another embodiment, pellets suitable for use may have a mean particle size of from about 0.01 μm to about 250 μm.

Another specific suitable formulation principle for use in the present context is formulation in a lipophilic environment such as, e.g., soft gelatin capsules. Vegicaps Soft from Scherer (a soft capsule technology based on carrageenan and starch. While this new dosage form is 100% plant-derived, it still offers all the key attributes of traditional soft gelatin capsules. These include a soft and flexible dosage form that provides ease of swallowing.) is a suitable example of such a formulation principle (please refer to http://www.rpscherer.de/page.php?pageID=94).

A further specific example of a suitable formulation comprises the compound according to the inventiontogether with vitamin E concentrate in soft or hard gelatin capsules. This formulation, in a modified form, is the basis of the commercial cyclosporine product, Neoral®, containing, among other things, corn oil-mono-di-triglycerides, polyoxyl 40 hydrogenated castor oil NF, DL-α-tocopherol USP (part of the vitamin E family), gelatin NF, glycerol, iron oxide black, propylene glycol USP, titanium dioxide USP, carmine, and alcohol in addition to cyclosporine.

Another specific example of a suitable formulation comprises the compound according to the invention together with ethanol, tocopherolethylene glycol 1000 succinate (TPGS), corn oil and wax in soft or hard gelatin capsules. This product can be a semi-solid or solid dosage form. The release rate of this formulation is dependent on erosion due to lipases in the intestine.

A further example of a suitable formulation comprises the formulation of a compound according to the invention together with ethanol, tocopherolethylene glycol 1000 succinate (TPGS), corn oil and polyglycolized glycerides (e.g. Gelucire) in soft or hard gelatin capsules. This product can be a semi-solid or solid dosage form. The release rate of this formulation is dependent on degradation due to lipases in the intestine.

A further example of a suitable formulation is an oral pulsed dose drug delivery system. This dosage form can be perceived as a modified form of the Schering Repetab tablets. A portion of the composition of the present invention is put in the core of a tablet.

The core can for example be made by conventional wet granulation or continuous granulation such as extrusion followed by compaction of the granulate into tablets. The core is then coated using an appropriate technology, preferably by airsuspension using an enteric coating polymer such as Eudragits.

The first releasing dose is compression coated on the core or air-suspension coated either with the enteric coat or on top of the enteric coat. In a embodiment of the invention, the first releasing dose is air-suspension coated with the enteric coat. In a further embodiment of the invention, the first releasing dose is compression coated on the core, in order to avoid release of the composition according to the invention prior to the degradation of the enteric coat, such degradation typically occurring at pH values higher than those found in the gastric ventricle; i.e. the degradation of the enteric coat typically occurs after passage of the gastric ventricle.

A further example of a suitable formulation is an oral sustained drug delivery system. A portion of the composition of the present invention is put in the core of a tablet.

The core can for example be made by conventional wet granulation or continuous granulation such as extrusion followed by compaction of the granulate into tablets. The core is coated using an appropriate technology, preferably by airsuspension using ethylcellulose and a hydrophilic excipient such as hydroxyl propyl cellulose (HPC).

The first releasing dose is compression coated on the core or air-suspension coated either with the enteric coat or on top of the enteric coat. In a preferred embodiment of the invention, the first releasing dose is air-suspension coated with the enteric coat. In a further embodiment of the invention, the first releasing dose is compression coated on the core, in order to avoid release of the composition according to the invention prior to the degradation of the enteric coat, such degradation typically occurring at pH values higher than those found in the gastric ventricle; i.e. the degradation of the enteric coat typically occurs after passage of the gastric ventricle.

A further example of a suitable formulation is obtained via crystal engineering, such as e.g. described in WO 03/080034, which is hereby incorporated by reference.

Accordingly, in another embodiment the composition of the invention comprises the novel salt in the form of micro crystals with hydrophilic surfaces. Furthermore, in another embodiment of the invention, the micro crystals are filmcoated directly, in order to achieve a sustained release formulation.

Another specific example of a suitable formulation comprises complexation of the salt according to the present invention with genuine cyclodextrins and cyclodextrin-derivatives (e.g. alkyl- and hydroxyalkyl-derivatives or sulfobutyl-derivatives). The complexation is achieved in accordance with well known methods. It is contemplated that such a complexation leads to a higher solubility and a higher dissolution rate of the composition according to the invention, compared to the composition prior to complexation. Furthermore, it is contemplated that such a complexation leads to a higher bioavailability of the composition according to the invention, compared to the composition prior to complexation. In specific embodiments, the invention relates to a controlled release pharmaceutical composition that may be administered one, two or more times daily, such as once, twice or three times daily. Furthermore, the composition may be designed so that it releases the fumaric acid ester relatively independent on pH, i.e. the release is not dependent on pH in the gastrointestinal tract. Examples of such compositions are e.g. compositions in the form of solid dosages forms (e.g. tablets, capsules, pellets, beads etc.) that are coated with a controlled release coating. Suitable materials for controlled release coatings are e.g. cellulose and cellulose derivatives including methylcellulose, ethylcellulose and cellulose acetate, or poly(ethylene-co-vinyl acetate), poly(vinyl chloride).

The release of the fumaric acid ester typically takes place in three steps from a composition coated with a diffusion controlled membrane:
i) firstly, water (from the GI tract) diffuses into the dosage form from the surroundings,
ii) secondly, at least some of the fumaric acid ester present in the dosage form dissolves by the action of water,
iii) the dissolved fumaric acid ester diffuses out of the dosage form and into the surroundings (i.e. the GI tract)

Other examples include e.g. matrix tablets or dosage form containing a multiplicity of units each in the form of a matrix system. The active substance is embedded in a matrix containing e.g. cellulose and cellulose derivatives including microcrystalline cellulose, hydroxypropyl methyl cellulose, hydroxypropyl cellulose and methylcellulose, povidone, poly(ethyleneoxide) (PEO), polyethylene glycol (PEG), poly(vinyl alcohol) (PVA), xanthan gum, carrageenan and other synthetic materials. Substances normally used as pharmaceutically acceptable excipients or additives may be added to a matrix composition.

Other examples of suitable compositions are e.g. hydrogels, i.e. monolithic systems wherein the active substance is embedded in a water-swellable network polymer. Materials suitable for use include e.g. hydrophilic vinyl and acrylic polymers, polysaccharides like alginates, and poly(ethylene oxide).

In specific embodiments, a composition according to the invention has a pH controlled release (also known as pH dependent release) of the fumaric acid ester. Normally, the release is designed so that only a small amount, if any, of the fumaric acid ester is released in the stomach (pH up to about 3), whereas the fumaric acid ester is released in the intestines (pH shifts to about 6-7). Such a pH dependent release can be obtained by providing a composition of the invention with an enteric coating (the whole composition or, if the composition is a multiparticulate composition, the individual units) or by providing a composition that releases the fumaric acid ester by a pH-dependent osmotic mechanism, or by employment of suitable enzymes.

Examples of suitable substances for use as enteric coating materials include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, poly acrylic methacrylic acid copolymers, shellac and vinyl acetate and crotonic acid copolymers, etc.

The compositions mentioned above having a pH independent release may also be formulated to release the fumaric acid ester e.g. by providing the composition with an outer layer of an enteric coating.

Furthermore, the compositions may be formulated in such a manner that an initial delay in release of the fumaric acid ester is obtained. Such a delay may be obtained e.g. by choosing an outermost coating that in a time-controlled manner degrades (e.g. erodes) and only when this outermost coating is eroded away, the release of the fumaric acid ester starts.

In one aspect of the invention, the compound according to the invention is formulated in a composition that enables a prolonged and/or a slow release of a fumaric acid ester as defined above. Examples of such compositions are for example described in PCT/DK2005/000648 which is hereby incorporated by reference.

In the present context, a controlled release composition is a composition that is designed to release the compound according to the invention in a prolonged, slow and/or delayed manner compared to the release of the commercially available product Fumaderm®, when tested under comparable conditions (e.g. for in vivo studies: dose equivalents, with or without standardized meal etc., or for in vitro studies: dose equivalents, dissolution test apparatus and working conditions including e.g. composition, volume and temperature of dissolution medium employed, rotation speed etc.).

The release in vivo may be tested by measuring the plasma concentration at predetermined time periods and thereby obtaining a plasma concentration versus time profile for the fumaric acid ester in question or, if relevant, a metabolite thereof. Furthermore, it is contemplated that metabolism already takes place within the gastrointestinal tract or during passage of the gastrointestinal mucosa, or upon first passage through the hepatic circulation.

Other tests may also be used to determine or to give a measure of the release of the active substance in vivo. Thus, animals (e.g. mice, rats, dogs etc.) may be used as a model. The animals receive the compositions under investigation and after specified periods of time, the animals are sacrificed and the content of the active ingredient (or metabolite thereof, if relevant) is determined in plasma or specific organs or extracted from the intestinal contents.

Another test involves the use of a specific segment of an animal intestine. The segment is placed in a suitable dissolution apparatus containing two compartments (a donor and a receiver) separated by the segment, and the composition under investigation is placed in a suitable medium in one compartment (the donor compartment). The composition will release the active substance that subsequently is transported across the intestinal segment. Accordingly, at suitable time intervals the concentration of the active substance (or, if relevant, the metabolite) is measured in the receiver compartment.

A person skilled in the art will be able to adapt the above-mentioned method to the specific composition.

With respect to in vitro methods, well-established methods are available, especially methods described by official monographs like e.g. United States Pharmacopeia (USP) or the European Pharmacopoeia. A person skilled in the art will know which method to choose and how to select the specific conditions to carry out the in vitro test. For instance, the USP prescribes in vitro tests be carried out at 37±1.0 such as 37±0.5 degrees Celsius/Centigrade. A suitable dissolution test is, for example for capsules, wherein the dissolution profile is determined as described in the United States Pharmacopoeia at 37° C. using a rotating basket at 100 rpm employing 0.1 N hydrochloric acid as dissolution medium during the first 2 hours of the test and then followed by 0.05 M phosphate buffer pH 6.5 as dissolution medium for the remaining test period, and, for example as described for tablets wherein the dissolution profile is determined as described in the United States Pharmacopoeia at 37° C. using a paddle dissolution apparatus at 100 rpm employing 0.1 N hydrochloric acid as dissolution medium during the first 2 hours of the test and then followed by 0.05 M phosphate buffer pH 6.5 as dissolution medium for the remaining test period.

As mentioned above, the in vivo release of the compound according to the invention is in one aspect of the invention prolonged, slow and/or delayed compared with the commercially available Fumaderm® composition.

With regard to the compound according to the invention, the term “prolonged” is in one embodiment intended to indicate that the active substance is released during a longer time period than Fumaderm® such as at least during a time period that is at least 1.2 times, such as, e.g., at least 1.5 times, at least 2 times, at least 3 times, at least 4 times or at least 5 times greater than that of Fumaderm®. Thus, if e.g. 100% of dimethylfumarate is released from Fumaderm® tablets 3 hours after the start of a suitable test, then 100% of the fumaric acid ester in a composition according to the invention is released at least 3.6 hours after the start of a suitable test.

With regard to the compound according to the invention the term “delayed” is in one embodiment intended to indicate that the release starts at a later point in time compared with that of Fumaderm® (such as at 30 min or more later such as, e.g., 45 min or more later, 1 hour or more later or 1.5 hours or more later, alternatively, that the initial release during the first 2 hours is much less compared with that of Fumaderm® (i.e. less than 80% w/w such as, e.g., less than 70% w/w, less than 60% w/w or less than 50% of that of Fumaderm®).

A useful composition comprising the compound according to the invention is a controlled release composition designed to be administered two or more times daily, such as e.g. described in PCT/DK2005/000648 which is hereby incorporated by reference.

In the following is given a description of various compositions according to the invention that are designed to obtain a suitable release of the monoalkyl fumaric acid ester (in the following fumaric acid ester). Based on the description above and handbooks within the field of controlled release of pharmaceutics, a person skilled in the art will know how to choose different formulation principles in order to achieve the required release profile.

Compositions Designed to be Administered Two or More Mimes Daily

pH Dependent Release

In the following is given a description of specific embodiments, wherein the fumaric acid ester is released depending on pH and wherein the release pattern is suitable for compositions that are administered two or more times daily. Examples of suitable formulation principles are e.g. compositions provided with an enteric coating or hydrogels of a type described by Zentner et al (U.S. Pat. No. 6,537,584) and Bae (U.S. Pat. No. 5,484,610), which hereby are incorporated by reference. Further examples of suitable formulation principles are e.g. compositions provided with a diffusion coating such as a controlled release diffusion coating, matrix particulates or matrix tablets, hydrogels, pulsed dose drug delivery systems, co-formulation with vitamin E concentrate or ethanol, TPGS, corn oil and wax etc., including any of the formulation principles mentioned above, optionally with an enteric coating.

Accordingly, one aspect the invention relates to a controlled release pharmaceutical composition for oral use comprising as an active substance a amino acid salt of a mono-(C₁-C₅)alkylester of fumaric acid, wherein the release of the fumaric acid ester—when subjected to an in vitro dissolution test employing 0.1 N hydrochloric acid as dissolution medium during the first 2 hours of the test and then 0.05 M phosphate buffer pH 6.5 or 6.8 as dissolution medium—is as follows:

within the first 2 hours after start of the test at the most about 15% w/w such as, e.g. at the most about 10% w/w, at the most about 5% w/w of the total amount of the fumaric acid ester is released, and/or

within the first 2 hours after start of the test at least about 1% w/w such as, e.g. at least about 2% w/w, at least about 3% w/w, or about 5% w/w of the total amount of the fumaric acid ester is released, and/or

within the first 3 hours after start of the test at the most about 35% w/w such as, e.g., from about 15% to about 35% w/w, from about 20% to about 30% w/w, or about 25% w/w of the total amount of the fumaric acid ester is released, and/or

within the first 3 hours after start of the test at the most about 90% w/w such as, e.g., from about 5% to about 90% w/w, from about 5% to about 85% w/w, from about 10% to about 80% w/w, from about 10% to about 70% w/w, from about 10% to about 65% w/w, from about 10% to about 60% wlw, from about 15% to about 50% w/w, from about 15% to about 35% w/w, from about 20% to about 30% w/w, or about 20% w/w, or about 25% w/w of the total amount of the fumaric acid ester is released, and/or

within the first 4 hours after start of the test at the most about 92% w/w such as, e.g., from about 10% to about 92% w/w, from about 20% to about 85% w/w, from about 20% to about 80% w/w, from about 20% to about 70% w/w, from about 25% to about 60% w/w, from about 25% to about 55% w/w, from about 30% to about 50% w/w, or about 35% w/w, or about 40% w/w, or about 45% w/w of the total amount of the fumaric acid ester is released, and/or

within the first 5 hours after start of the test at the most about 94% w/w such as, e.g., from about 15% to about 94% w/w, from about 25% to about 90% w/w, from about 30% to about 85% w/w, from about 35% to about 80% w/w, from about 35% to about 75% w/w, from about 40% to about 70% w/w, from about 45% to about 70% w/w, from about 55% to about 70% w/w, from about 60% to about 70% w/w, or about 45% w/w, or about 50% w/w, or about 55% w/w, or about 60% w/w, or about 65% w/w of the total amount of the fumaric acid ester is released, and/or

within the first 6 hours after start of the test at the most about 60% w/w such as, e.g., from about 30% to about 60% w/w, from about 40% to about 55% w/w, or about 50% w/w of the total amount of the fumaric acid ester contained in the composition is released, and/or

within the first 6 hours after start of the test at the most about 95% w/w such as, e.g., from about 35% to about 95% w/w, from about 40% to about 90% w/w, from about 45% to about 85% w/w, from about 50% to about 85% w/w, from about 55% to about 85% w/w, from about 60% to about 85% w/w, from about 65% to about 85% w/w, from about 70% to about 85% w/w, from about 75% to about 85% w/w, or about 65% w/w, or about 70% w/w, or about 75% w/w, or about 80% w/w of the total amount of the fumaric acid ester contained in the composition is released, and/or

within the first 7 hours after start of the test at the most about 98% w/w such as, e.g., from about 45% to about 98% w/w, from about 50% to about 98% w/w, from about 55% to about 98% w/w, from about 60% to about 98% w/w, from about 65% to about 98% w/w, from about 70% to about 98% w/w, from about 75% to about 95% w/w, from about 80% to about 95% w/w, from about 85% to about 95% w/w, or about 75% w/w, or about 80% w/w, or about 85% w/w, or about 90% w/w of the total amount of the fumaric acid ester contained in the composition is released, and/or

within the first 9 hours after start of the test at the most about 85% w/w such as, e.g., from about 50% to about 85% w/w, from about 60% to about 80% w/w, or about 75% w/w of the total amount of the fumaric acid ester contained in the composition is released, and/or

within the first 9 hours after start of the test at the most about 99% w/w such as, e.g., from about 60% to about 99% w/w, from about 70% to about 99% w/w, from about 80% to about 99% w/w, from about 90% to about 99% w/w, or about 95% w/w of the total amount of the fumaric acid ester contained in the composition is released, and/or

within the first 12 hours after start of the test at least about 80% w/w such as, e.g., about 80% w/w or more, about 85% w/w or more, about 90% w/w or more or about 95% w/w or more of the total amount of the fumaric acid ester contained in the composition is released.

Compositions Designed to be Administered Once Daily

pH Dependent Release

In the following is given a description of specific embodiments, wherein the fumaric acid ester is released dependently of pH and wherein the release pattern is suitable for compositions that are administered once daily. Examples of suitable formulation principles are e.g. compositions provided with an enteric coating or hydrogels of a type described by Zentner et al (U.S. Pat. No. 6,537,584) and Bae (U.S. Pat. No. 5,484,610). Further examples of suitable formulation principles are e.g. compositions provided with a diffusion coating such as a controlled release diffusion coating, matrix particulates or matrix tablets, hydrogels, pulsed dose drug delivery systems, co-formulation with vitamin E concentrate or ethanol, TPGS, corn oil and wax etc., including any of the formulation principles mentioned above, optionally with an enteric coating.

Accordingly, in one aspect the invention relates to a controlled release pharmaceutical composition for oral use comprising as an active substance a amino acid salt of a mono-(C₁-C₅)alkylester of fumaric acid, wherein the release of the fumaric acid ester—when subjected to an in vitro dissolution test employing 0.1 N hydrochloric acid as dissolution medium during the first 2 hours of the test and then 0.05 M phosphate buffer pH 6.5 or 6.8 as dissolution medium—is as follows:

within the first 2 hours after start of the test at the most about 15% w/w such as, e.g., at the most about 10% w/w or at the most about 5% w/w of the total amount of the fumaric acid ester is released, and/or

within the first 2 hours after start of the test at least about 1% w/w such as, e.g. at least about 2% w/w, at least about 3% w/w, or about 5% w/w of the total amount of the fumaric acid ester is released, and/or

within the first 4 hours after start of the test at the most about 90% w/w such as, e.g., from about 5% to about 90% w/w, from about 5% to about 85% w/w, from about 10% to about 80% w/w, from about 10% to about 70% w/w, from about 10% to about 65% w/w, from about 10% to about 60% w/w, from about 15% to about 50% w/w, from about 15% to about 35% w/w, from about 20% to about 30% w/w, or about 20% w/w, or about 25% w/w of the total amount of the fumaric acid ester is released, and/or

within the first 4.5 hours after start of the test at the most about 35% w/w such as, e.g., from about 15% to about 35% w/w, from about 20% to about 30% w/w, or about 25% w/w of the total amount of the fumaric acid ester is released, and/or

within the first 5 hours after start of the test at the most about 92% wlw such as, e.g., from about 10% to about 92% w/w, from about 20% to about 85% w/w, from about 20% to about 80% w/w, from about 20% to about 70% w/w, from about 25% to about 60% w/w, from about 25% to about 55% w/w, from about 30% to about 50% w/w, or about 35% w/w, or about 40% w/w, or about 45% w/w of the total amount of the fumaric acid ester is released, and/or

within the first 6 hours after start of the test at the most about 94% w/w such as, e.g., from about 15% to about 94% w/w, from about 25% to about 90% w/w, from about 30% to about 85% w/w, from about 35% to about 80% w/w, from about 35% to about 75% w/w, from about 40% to about 70% w/w, from about 45% to about 70% w/w, from about 55% to about 70% w/w, from about 60% to about 70% w/w, or about 45% w/w, or about 50% w/w, or about 55% w/w, or about 60% w/w, or about 65% w/w of the total amount of the fumaric acid ester is released, and/or

within the first 7 hours after start of the test at the most about 95% w/w such as, e.g., from about 35% to about 95% w/w, from about 40% to about 90% w/w, from about 45% to about 85% w/w, from about 50% to about 85% w/w, from about 55% to about 85% w/w, from about 60% to about 85% w/w, from about 65% to about 85% w/w, from about 70% to about 85% w/w, from about 75% to about 85% w/w, or about 65% w/w, or about 70% w/w, or about 75% w/w, or about 80% w/w of the total amount of the fumaric acid ester contained in the composition is released, and/or

within the first 9 hours after start of the test at the most about 98% w/w such as, e.g., from about 45% to about 98% w/w, from about 50% to about 98% w/w, from about 55% to about 98% w/w, from about 60% to about 98% w/w, from about 65% to about 98% w/w, from about 70% to about 98% w/w, from about 75% to about 95% w/w, from about 80% to about 95% w/w, from about 85% to about 95% w/w, or about 75% w/w, or about 80% w/w, or about 85% w/w, or about 90% w/w of the total amount of the fumaric acid ester contained in the composition is released, and/or

within the first 9 hours after start of the test at the most about 60% w/w such as, e.g., from about 30% to about 60% w/w, from about 40% to about 55% w/w, or about 50% w/w of the total amount of the fumaric acid ester contained in the composition is released, and/or

within the first 12 hours after start of the test at the most about 99% w/w such as, e.g., from about 60% to about 99% w/w, from about 70% to about 99% w/w, from about 80% to about 99% w/w, from about 90% to about 99% w/w, or about 95% w/w of the total amount of the fumaric acid ester contained in the composition is released, and/or

within the first 13.5 hours after start of the test at the most about 85% w/w such as, e.g., from about 50% to about 85% w/w, from about 60% to about 80% w/w, or about 75% w/w of the total amount of the fumaric acid ester contained in the composition is released, and/or

within the first 18 hours after start of the test at least about 80% w/w such as, e.g., about 80% w/w or more, about 85% w/w or more, about 90% w/w or more or about 95% w/w or more of the total amount of the fumaric acid ester contained in the composition is released, and/or

the total amount of the fumaric acid ester contained in the composition is released within the first 18 hours after start of the test.

Typically, as described above, the compositions according to the invention are designed to deliver the active substance (i.e. the monoalkylester of fumaric acidwhich in turn is metabolised to fumaric acid and, which subsequently is subjected to a rapid elimination process) in a prolonged manner. Apart from the characteristic in vitro release patterns described herein, such a prolonged release is reflected in the pharmacokinetic parameters obtained after a clinical study as well. Accordingly, it is contemplated that the cₘₐₓ of the monoalkylester of fumaric acid (which appears in the plasma upon hydrolysis or metabolism of the dialkylester administered) is of the same order of magnitude as previously described in the literature provided that similar or equivalent dose is administered (i.e. e.g. a cₘₐₓ of monomethylfumarate in a range of from about 0.4 to about 2.0 mg/l). However, in order to avoid many frequent daily administrations (2-4 tablets 1-3 times daily) it is an aim to prolong the time period where the concentration is within the therapeutic window. Accordingly, it is contemplated that W₅₀ (i.e. the time period in which the plasma concentration is 50% of cₘₐₓ or more) is prolonged compared to the marketed treatment with at least 10% such as, e.g. at least 20%, at least 30%, at least 40% or at least 50%. A suitable W₅₀ is believed to be at least 2 hours such as in a range of from about 2 to about 15 hours or from about 2.5 to about 10 hours or from about 3 to about 8 hours.

Furthermore, it is contemplated that a controlled release composition according to the invention may lead to a reduced interindividual and/or intraindividual variation in the plasma profile and to a reduced dependency on whether the composition is taken together with or without food (a reduced variation of the plasma concentration profile of monomethylfumarate when the pharmaceutical composition is administered with or without concomitant food intake), compared to e.g. Fumaderm® or compared to the composition of the invention in an immediate release form. Therefore, the controlled release composition according to the invention may lead to a reduced frequency of dosing and/or a reduced average total daily dose.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are described. All publications mentioned herein are incorporated herein by reference to disclose and describe the methods and/or materials in connection with which the publications are cited. It must be noted that as used herein and in the appended claims, the singular forms “a”, “an”, and “the” include plural referents unless the context clearly dictates otherwise. The patents and publications discussed herein are provided solely for their disclosure prior to the filing date of the present application. Nothing herein is to be construed as an admission that the present invention is not entitled to antedate such patent or publication by virtue of prior invention. Further, the dates of publication provided may be different from the actual publication dates which may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. The figures shown herein are not necessarily drawn to scale, with some components and features being exaggerated for clarity.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### EXAMPLE 1

### Preparation of (S)-2,6-dihydro-2,6-diaminohexanal-(E)-methoxy-4-oxobut-2-enoate(lysine monomethylfumarate)

Lysine (Fluka, 62840) and monomethyl fumarate, MMF (Sigma-Aldrich, 651419, CAS 2756-87-8) in equimolar amounts (0.05 M) were dissolved in 3.5 mL of water. The mixture was stirred and heated until dissolution of all solid material. The solution was transferred to a beaker with 600 mL of acetone, which resulted in formation of a white precipitate. A fine-grained, dusty, white powder formed after suction filtration and drying in an electrical oven set at 40° C. No specific melting point was observed, which indicates that the product compound was amorphous. The amorphous state was confirmed by x-ray powder crystallography. UV-spectrophotometry was used to check the ratio of lysine to MMF (208 nm) in the product. A value for the molar mass was estimated by titration.

### EXAMPLE 2

### Preparation of 2-hydro-amino-((E)-methoxy-4-oxobut-2-enoate)-3-hydroxybutanoic acid (threonine monomethylfumarate)

Threonine (Fluka, 89180) and MMF (Sigma-Aldrich, 651419, CAS 2756-87-8) in equimolar amounts (0.025 M) were dissolved in 4.0 mL of water. The mixture was stirred and heated until dissolution of all solid material. The solution was transferred to a beaker with 800 mL of acetone, which resulted in formation of a white precipitate. A white powder formed after suction filtration and drying in an electrical oven set at 40° C. No specific melting point was observed, which indicates that the product compound was amorphous. The amorphous state was confirmed by x-ray powder crystallography. UV-spectrophotometry was used to check the ratio of threonine to MMF (208 nm) in the product. A value for the molar mass was estimated by titration.

### EXAMPLE 3

### Preparation of hydro-pyrrolidine-((E)-methoxy-4-oxobut-2-enoate)-2-carboxylic acid (proline monomethylfumarate)

Proline (Fluka, 82710) and MMF (Sigma-Aldrich, 651419, CAS 2756-87-8) in equimolar amounts (0.025 M) were dissolved in 4.0 mL of water. The mixture was stirred and heated until dissolution of all solid material. The solution was transferred to a beaker with 800 mL of acetone, which resulted in formation of a white precipitate. A white powder formed after suction filtration and drying in an electrical oven set at 40° C. No specific melting point was observed, which indicates that the product compound was amorphous. The amorphous state was confirmed by x-ray powder crystallography. UV-spectrophotometry was used to check the ratio of proline to MMF (208 nm) in the product. A value for the molar mass was estimated by titration.

### EXAMPLE 4

### Preparation of (S)-2-hydro-amino-((E)-methoxy-4-oxobut-2-enoate)-3-(1H-imidazol-5-yl)propanoic acid (histidine monomethylfumarate)

Histidine (Fluka, 53320) and MMF (Sigma-Aldrich, 651419, CAS 2756-87-8) in equimolar amounts (0.025 M) were dissolved in 4.0 mL of water at 60-70° C. and the solution was stirred until dissolution of all solid material. The solution was transferred to a beaker with 570 mL of ice-cold acetone at 0° C. A white and sticky material precipitated was formed following this treatment. An amorphous and transparent solid material was formed after suction filtration and drying in an electrical oven at 40° C. for 72 hours. No specific melting point was observed, which indicates that the product compound was amorphous. The amorphous state was confirmed by x-ray powder crystallography. UV-spectrophotometry was used to check the ratio of histidine to MMF (208 nm) in the product. A value for the molar mass was estimated by titration.

### EXAMPLE 5

### Preparation of 2-hydro-((E)-methoxy-4-oxobut-2-enoate)-aminopropanoic acid (alanine monomethylfumarate)

Alanine (Fluka, 05129) and MMF (Sigma-Aldrich, 651419, CAS 2756-87-8) in equimolar amounts (0.025 M) were dissolved in 4.0 mL of water and the pH-value was adjusted to 7-8. The solution was heated to 65-70° C. and the solution was stirred until dissolution of all solid material. The solution was transferred to a beaker with 500 mL of acetone, which resulted in the formation of a white precipitate. A white powder formed after suction filtration and drying in an electrical oven set at 40° C. No specific melting point was observed, which indicates that the product compound was amorphous. The amorphous state was confirmed by x-ray powder crystallography. UV-spectrophotometry was used to check the ratio of alanine to MMF (208 nm) in the product. A value for the molar mass was estimated by titration.

### EXAMPLE 6

### Preparation of 2-hydro-amino-((E)-methoxy-4-oxobut-2-enoate)-acetic acid (glycine monomethylfumarate)

Glycine (Fluka, 50049, CAS 56-40-6) and MMF (Sigma-Aldrich, 651419, CAS 2756-87-8) in equimolar amounts (0.025 M) were dissolved in 3.75 mL of water. The mixture was stirred and heated until dissolution of all solid material. The solution was transferred to a beaker with 400 mL of acetone, which resulted in formation of a white precipitate. A white powder formed after suction filtration and drying in an electrical oven set at 40° C. No specific melting point was observed, which indicates that the product compound was amorphous. However, the compound exhibited a point of decomposition that was observed at approx. 200° C. The amorphous state was confirmed by x-ray powder crystallography. UV-spectrophotometry was used to check the ratio of glycine to MMF (208 nm) in the product. A value for the molar mass was estimated by titration.

### EXAMPLE 7

### Preparation of 2-hydro-amino-((E)-methoxy-4-oxobut-2-enoate)-3-hydroxypropanoic acid (serine monomethylfumarate)

Serine (Fluka, 84960, CAS 56-45-1) and MMF (Sigma-Aldrich, 651419, CAS 2756-87-8) in equimolar amounts (0.025 M) were dissolved in 5.0 mL of water and the pH-value was adjusted by sodium hydroxide (Fluka, 71689, CAS 1310-73-2) until dissolution of all solid material. The mixture was stirred and heated until dissolution of all solid material. The solution was transferred to a beaker with 400 mL of acetone, which resulted in formation of a white precipitate. A white powder formed after suction filtration and drying in an electrical oven set at 40° C. Two melting points were observed, which corresponded to those of the reactants. UV-spectrophotometry was used to check the ratio of serine to MMF (208 nm) in the product. A value for the molar mass was estimated by titration.

### EXAMPLE 8

### Preparation of 2-hydro-amino-((E)-methoxy-4-oxobut-2-enoate)-5-guanidinopentanoic acid (arginine monomethylfumarate)

Arginine (Fluka, 11010, CAS 74-79-3) and MMF (Sigma-Aldrich, 651419, CAS 2756-87-8) in equimolar amounts (0.025 M) were dissolved in 4.0 mL of water. The mixture was stirred and heated until dissolution of all solid material. The solution was transferred to a beaker with 500 mL of acetone, which resulted in formation of a white sticky material. The white sticky material remained after suction filtration and drying in an electrical oven set at 40° C. UV-spectrophotometry was used to check the ratio of arginine to MMF (208 nm) in the product. An approximate value for the molar mass was estimated by titration.

### EXAMPLE 9

### Preparation of 2-hydro-amino-((E)-methoxy-4-oxobut-2-enoate)-3-mercaptopropanoic acid (cystein monomethylfumarate)

Cystein (Fluka, 30089, CAS 52-90-4) and MMF (Sigma-Aldrich, 651419, CAS 2756-87-8) in equimolar amounts (0.025 M) were dissolved in 3.5 mL of water at 60-70° C. and the solution was stirred until dissolution of all solid material. The solution was transferred to a beaker with 420 mL of acetone, which resulted In the precipitation of a white and sticky material. An amorphous and transparent solid material was formed after suction filtration and drying in an electrical oven at 60° C. After drying, the material was hard and transparent. Heating to temperatures above 110° C. caused degradation, as observed by a yellow colouring of the product. No specific melting point was observed, which indicates that the product compound was amorphous. The amorphous state was confirmed by x-ray powder crystallography. UV-spectrophotometry was used to check the ratio of cystein to MMF (208 nm) in the product. A value for the molar mass was estimated by titration.

### EXAMPLE 10

### Preparation of 2,4-dihydro-2,4-diamino-((E)-methoxy-4-oxobut-2-enoate)-4-oxobutanoic acid (asparagine monomethylfumarate)

Asparagine (Fluka, 11150, CAS 70-47-3) and MMF (Sigma-Aldrich, 651419, CAS 2756-87-8) in equimolar amounts (0.025 M) were dissolved in 3.5 mL and the pH-value was adjusted to 7 by addition of sodium hydroxide (Fluka, 71689, CAS 1310-73-2). The solution was heated to 85° C., and the solution was stirred until dissolution of all solid material. The solution was transferred to a beaker with 1250 mL of acetone, which resulted in the formation of a white precipitate. A white powder formed after suction filtration and drying in an electrical oven set at 40° C. No specific melting point was observed, which indicates that the product compound was amorphous. The amorphous state was confirmed by x-ray powder crystallography. UV-spectrophotometry was used to check the ratio of asparagine to MMF (208 nm) in the product. A value for the molar mass was estimated by titration.

### EXAMPLE 11

### Measuring intestinal permeability using Caco-2 cell monolayers.

Material and Methods

Cell Culture:

Caco-2 cells are cultured in Dulbecco's modified Eagle's medium (DMEM), containing 10% fetal bovine serum (FBS), 1 mM sodium pyruvate, 100 pM non-essential amino acids, 2 mM L-glutamine, 100 U/mL penicillin, and 100 pg/mL streptomycin. Cells are cultured in 175 cm² flasks (Costar USA). For transport studies, cells are harvested from the flasks with a trypsin-EDTA (0.25% (w/v)−1 mM EDTA) solution (Gibco Life Technologies) and are seeded at a density of 60,000 cells/cm² on collagen-coated Transwell® polycarbonate filters (0.4 μm pore size, 1.13 cm² surface area) (Costar 3401). Cells on flasks or Transwell® are cultured at 37° C. in a humidified atmosphere containing 5% CO₂. Culture media is changed every other day for 10 days, and daily afterwards. Cell monolayers are used between 21 and 28 days post-seeding.

Permeability Assay:

The permeability assay buffer (PAB) is Hank's balanced salts solution containing 15 mM D(+)glucose and 10 mM HEPES, pH 7.3±0.1 The quality control of cell monolayers to be used in the permeability assays is conducted in two steps. The first step consists of certifying the suitability of the batch of cells and the second step consist of testing each monolayer in the entire seeding. To certify a batch (seeding) of cells, a subgroup of randomly-selected monolayers is tested with respect to transepithelial electrical resistance (TEER) measurements and permeability to several control compounds. TEER values are measured in PAB using an epithelial voltammeter with an Endohm-12 electrode (World Precision Instruments). The control compounds used to assess the suitability of a batch of cells are digoxin, lucifer yellow, atenolol, and propranolol. For each monolayer tested the TEER value and the permeability coefficient for the different control compounds have to be within a specified range before a given batch of cells may be certified as “acceptable”.

Pre-Experiment Batch Acceptance Criteria:

Atenolol Papp (×10⁻⁶ cm/s)<0.5

Propranolol Papp (×10⁻⁶ cm/s)15-25

Lucifer Yellow Papp (×10⁻⁶ cm/s)<0.4

Digoxin Ratio Papp (B-A)/Papp(A-B)>3

Following the acceptance of a batch, the TEER value of each monolayer intended to be used in permeability studies is measured prior to inclusion in the study. Monolayers with TEER values in the 450-650 Ω.cm² range are included in the permeability study, and those with TEER values outside this range are discarded.

Test Method:

Uni-directional permeability of pro-drug (compound according to the invention) and conversion assessment of pro-drug to drug (corresponding fumaric acid ester) is performed as follows:

Caco-2 cell monolayers are grown to confluence on collagen-coated Transwell® polycarbonate filters as outlined above, and used 21 to 28 days post-seeding. The permeability assay buffer is Hank's Balanced Salt Solution containing 10 mM HEPES and 15 mM glucose at a pH of 7.4. The dosing solution concentration is e.g. 100 μM of the pro-drug in assay buffer. The receiver (basolateral) side contains 1% bovine serum albumin (BSA) in modified Hank's humidified incubator. Each determination is performed in duplicate (in duplicate wells). At t=90 minutes, the receiver as well as the donor sides are sampled, and both samples are analyzed with respect to the amount of pro-drug and the amount of converted drug (assessing the apical-to-basolateral transport of pro-drug (A to B)). Lucifer yellow flux is also measured for each monolayer after being subjected to the test articles to ensure that no damage is inflicted to the cell monolayers during the flux period. Both receiver and donor samples are assayed by liquid chromatography (LC)/mass spectrometer (MS) with an appropriate standard curve.

Analytical:

All samples are analyzed by LC/MS using a PE SCIEX API 150 or API 2000 mass spectrometer. The chromatographic system consists of two Perkin Elmer Series 200 micro LC pumps and a Perkin Elmer Series 200 autosampler.

Permeability Calculation:

The apparent permeability (Papp) and percent recovery can be calculated according to the following equation: Papp=(dCᵣ/dt)×Vᵣ/(A×C₀) where, dCᵣ/dt is the cumulative concentration in the receiver compartment versus time in μM s⁻¹. Vᵣ is the volume of the receiver compartment (e.g. 1.5 cm³). A is the area of the cell monolayer (1.13 cm² for 12-well Transwell). C₀ is the concentration of the dosing solution in μM. A linear fit of the cumulative concentration versus time is made to determine dCᵣ/dt. The origin is not included in the fit.

1. A compound of the general formula (I) 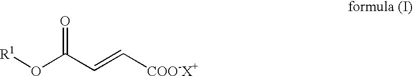
wherein
R¹ is: C₁₋₅alkyl and
X⁺ is a protonated form of an amino acid, and any enantiomers or racemic mixtures thereof.

2. The compound according to claim 1 selected from the group consisting of
amino acid salts of monomethylester of fumaric acid,
amino acid salts of monoethylester of fumaric acid,
amino acid salts of monopropylester of fumaric acid,
amino acid salts of monobutylester of fumaric acid, and
amino acid salts of monopentylester of fumaric acid.

3. The compound according to claim 1, wherein the amino acid is selected from the group consisting of natural amino acids.

4. The compound according to claim 3, wherein the amino acid is selected from the group consisting of lysine, arginine, glutamine, histidine, ornithine and tryptophan.

5. The compound according to claim 1, which is an amino acid salt of the monomethylester of fumaric acid.

6. The compound according to claim 1, which compound is selected from the group consisting of:
(S)-2-hydro-2,6-diaminohexanal-(E)-methoxy-4-oxobut-2-enoate(lysine monomethylfumarate),
(S)-6-hydro-2,6-diaminohexanal-(E)-methoxy-4-oxobut-2-enoate(lysine monomethylfumarate),
2-hydro-amino-((E)-methoxy-4-oxobut-2-enoate)-3-hydroxybutanoic acid (threonine monomethylfumarate),
hydro-pyrrolidine-((E)-methoxy-4-oxobut-2-enoate)-2-carboxylic acid (proline monomethylfumarate),
(S)-2-hydro-amino-((E)-methoxy-4-oxobut-2-enoate)-3-(IH-imidazol-5-yl)propanoic acid (histidine monomethylfumarate),
2-hydro-((E)-methoxy-4-oxobut-2-enoate)-aminopropanoic acid (alanine monomethylfumarate),
2-hydro-amino-((E)-methoxy-4-oxobut-2-enoate)-acetic acid (glycine monomethylfumarate),
2-hydro-amino-((E)-methoxy-4-oxobut-2-enoate)-3-hydroxypropanoic acid (serine monomethylfumarate),
2-hydro-amino-((E)-methoxy-4-oxobut-2-enoate)-5-guanidinopentanoic acid (arginine monomethylfumarate),
2-hydro-amino-((E)-methoxy-4-oxobut-2-enoate)-3-mercaptopropanoic acid (cystein monomethylfumarate),
2-hydro-2,4-diamino-((E)-methoxy-4-oxobut-2-enoate)-4-oxobutanoic acid (asparagine monomethylfumarate), and
4-hydro-2,4-diamino-((E)-methoxy-4-oxobut-2-enoate)-4-oxobutanoic acid (asparagine monomethylfumarate).

7. A compound according to claim 5, which is a lysine salt of the monomethylester of fumaric acid.

8. A composition comprising a compound according to claim 1 in combination with di(C₁₋₅)alkylester of fumaric acid,

9. A composition comprising a compound according to claim 1 in combination with a mono(C₁₋₅)alkylester of fumaric acid, optionally in the form of a pharmaceutically acceptable salt.

10-18. (canceled)

19. A pharmaceutical composition comprising a compound as defined according to claim 1.

20. The pharmaceutical composition according to claim 19 in the form of a controlled release composition.

21. A method of treating and/or preventing one or more conditions selected from the group consisting of psoriasis, psoriatic arthritis, neurodermatitis, atopic dermatitis, inflammatory bowel disease, autoimmune diseases, pain, organ transplantation (prevention of rejection), sarcoidosis, necrobiosis lipoidica, granuloma annulare, lupus nephritis, myasthenia gravis, uveitis, refractory uveitis, vernal conjunctivitis, pemphigus vulgaris, and/or scleroderma, which method comprises administering orally to a patient in need thereof, an effective dosage of a compound according to claim 1.

22. The method according to claim 21, wherein the autoimmume disease is multiple sclerosis.

23. The method according to claim 21, wherein the condition is psoriasis.

24. The method according to claim 21, wherein the condition is psoriatic arthritis.
